(19) United States
(12) Patent Application Publication
(10) Pub. No.: US 2002/0056151 A1
(43) Pub. Date: May 9, 2002

(54) Receptors for peptides from insects

(76) Inventors: Horst-Peter Antonicek, Bergisch Gladbach (DE); Gabi Friedrich, Leverkusen (DE); Thomas Schulte, Koln (DE)

Correspondence Address: BAYER CORPORATION, PATENT DEPARTMENT, 100 BAYER ROAD, PITTSBURGH, PA, 15205, US

(21) Appl. No.: 09/804,551

(22) Filed: Mar. 12, 2001

(30) Foreign Application Priority Data
Mar. 18, 2000 (DE) 10013618.4

(51) Int. Cl. A01H 5/00; C07H 21/04; C12P 21/02; C12N 5/04; C07K 14/705

(52) U.S. Cl. 800/279; 530/350; 536/23.5; 435/320.1; 435/410; 435/69.1

(57) ABSTRACT

The invention relates to polypeptides having the biological activity of peptide receptors, and to nucleic acids encoding these polypeptides, and in particular to their use for finding active compounds for crop protection.

The invention relates to polypeptides having the biological activity of peptide receptors, and to nucleic acids encoding these polypeptides, and in particular to their use for finding active compounds for crop protection.

Neuronal or endocrine peptides from insects are important target proteins for the development of novel insecticides, since such peptides regulate most of the important key functions, such as, for example, embryonal and postembryonal development, homeostasis, osmoregulation or muscle activity, in insects (see Gäde et al., 1997a; Osborne, 1996). The biological action of these peptides is mediated by binding to specific receptor proteins in insect cells. Many of these endocrines or neuronal peptides interact with G-protein-coupled receptors (GPCRs; King and Wilson, 1999) which, after binding of a corresponding peptide ligand, activate heterotrimeric G-proteins (Vanden Broeck et al., 1997). Agonists or antagonists of peptide receptors may, for example, interfere with normal insect development, with growth, behaviour or homeostasis, thus representing novel insect-specific and receptor-specific insecticides. These agonists or antagonists of peptide receptors can either be derived from the natural peptides or have an entirely novel chemical structure.

Since the isolation of the neuropeptide proctolin from preparations of insect muscles (Brown and Starratt, 1975), a large number of peptides from various insect species have been isolated and characterized (see Vanden Broeck et al., 1997; Osborne, 1996), for reviews). Substantial progress has been made in particular in the elucidation of the amino acid sequence of such peptides and in the elucidation of the biological function in the corresponding insect species. Thus, for example, peptides which can regulate the biosynthesis of juvenile hormones (allatotropines and allatostatines; Tobe et al., 1994), insulin-like peptides (Lagueux et al., 1990), peptides which regulate water homeostasis (Coast, 1998) or peptides which can control muscle activity (Holman, 1986; for a review, see Gäde , 1997b) have been isolated from various species. The biological functions of the peptides can be examined in various tests in which, for example, muscle activity (Holman et al., 1991) or the excretion of water and electrolytes (Ramsey, 1954) is measured.

Whereas a large number of peptides have been isolated, their structure elucidated and the amino acid sequence described, in insects only few receptors are known which are capable of binding endocrine or neuronal peptides (Reagan, 1994; Osborne, 1996; Birgul et al., 1999). It is therefore of great practical importance to provide peptide-binding receptors from insects, for example in the search for novel insecticides.

The present invention is therefore based, in particular, on the object of providing receptors from insects, hereinbelow referred to as receptors, which are capable of binding to endocrine or neuronal peptides from insects and which, by binding to the peptides, are capable of mediating the biological functions of these peptides, and of providing assay systems based thereon with a high throughput of test compounds (High Throughput Screening Assays; HTS Assays).

This object is achieved by providing polypeptides having at least one biological activity of a peptide receptor and comprising an amino acid sequence having at least 70% identity, preferably at least 80% identity, particularly preferably at least 90% identity, very particularly preferably at least 95% identity, with a sequence of SEQ ID NO:2, 4, 6, 8, 10, 12, 14, 16, 18, 20, 22, 24, 26, 28, 30, 32, 34, 36, 38, 40, 42, 44 or 46 over a length of at least 20, preferably at least 25, particularly preferably at least 30 consecutive amino acids, and very particularly preferably over their full length.

The degree of identity of the amino acid sequences is preferably determined using the program GAP from the program package GCG, Version 9.1, with standard settings (Devereux et al., 1984).

The term “polypeptides” as used in the present context not only relates to short amino acid chains which are usually referred to as peptide oligopeptides or oligomers, but also to longer amino acid chains which are usually referred to as proteins. It encompasses amino acid chains which can be modified either by natural processes, such as post-translational processing, or by chemical prior-art methods. Such modifications may occur at various sites and repeatedly in a polypeptide, such as, for example, on the peptide backbone, on the amino acid side chain, on the amino and/or the carboxyl terminus. For example, they encompass acetylations, acylations, ADP-ribosylations, amidations, covalent linkages to flavins, haem-moieties, nucleotides or nucleotide derivatives, lipids or lipid derivatives or phosphatidylinositol, cyclizations, disulphide bridge formations, demethylations, cystine formations, formylations, gamma-carboxylations, glycosylations, hydroxylations, iodinations, methylations, myristoylations, oxidations, proteolytic processings, phosphorylations, selenoylations and tRNA-mediated amino acid additions.

The polypeptides according to the invention may exist in the form of “mature” proteins or parts of larger proteins, for example as fusion proteins. They can furthermore exhibit secretion or leader sequences, pro-sequences, sequences which allow simple purification, such as multiple histidine residues, or additional stabilizing amino acids.

The polypeptides according to the invention need not constitute complete receptors, but may also be fragments thereof, as long as they still have at least one biological activity of the complete receptors. Polypeptides which, compared to receptors consisting of the polypeptides according to the invention having an amino acid sequence of SEQ ID NO:2, 4, 6, 8, 10, 12, 14, 16, 18, 20, 22, 24, 26, 28, 30, 32, 34, 36, 38, 40, 42, 44 or 46, have an activity which is increased or reduced by 50%, are still considered to be in accordance with the invention. The polypeptides according to the invention need not be deducible from Drosophila melanogaster receptors. Polypeptides which are also considered as being in accordance with the invention are those which correspond to receptors of, for example, the following invertebrates, or fragments thereof which can still exert the biological activity of these receptors: insects, nematodes, arthropods, molluscs.

In comparison to the corresponding region of naturally occurring receptors, the polypeptides according to the invention can have deletions or amino acid substitutions, as long as they still exert at least one biological activity of the complete receptors. Conservative substitutions are preferred. Such conservative substitutions comprise variations in which one amino acid is replaced by another amino acid from the following group:

1. small aliphatic residues, non-polar or of little polarity: Ala, Ser, Thr, Pro and Gly;

2. polar negatively charged residues and their amides: Asp, Asn, Glu and Gln;

3. polar positively charged residues: His, Arg and Lys;

4. large aliphatic non-polar residues: Met, Leu, Ile, Val and Cys; and

5. aromatic residues: Phe, Tyr and Trp.

Preferred conservative substitutions are shown in the list below:

| Original residue | Substitution |
|---|---|
| Ala | Gly, Ser |
| Arg | Lys |
| Asn | Gln, His |
| Asp | Glu |
| Cys | Ser |
| Gln | Asn |
| Glu | Asp |
| Gly | Ala, Pro |
| His | Asn, Gln |
| Ile | Leu, Val |
| Leu | Ile, Val |
| Lys | Mg, Gln, Glu |
| Met | Leu, Tyr, Ile |
| Phe | Met, Leu, Tyr |

| Original residue | Substitution |
|---|---|
| Ser | Thr |
| Thr | Ser |
| Trp | Tyr |
| Tyr | Trp, Phe |
| Val | Ile, Leu |

The term “biological activity of a peptide receptor” as used in the present context means binding of a peptide to the receptor.

Preferred embodiments of the polypeptides according to the invention are Drosophila melanogaster receptors which have the amino acid sequence of SEQ ID NO:2, 4, 6, 8, 10, 12, 14, 16, 18, 20, 22, 24, 26, 28, 30, 32, 34, 36, 38, 40, 42, 44 or 46.

The present invention also provides nucleic acids which encode the polypeptides according to the invention.

The nucleic acids according to the invention are, in particular, single-stranded or double-stranded deoxyribonucleic acids (DNA) or ribonucleic acids (RNA). Preferred embodiments are fragments of genomic DNA which may contain introns, and cDNAs.

Preferred embodiments of the nucleic acids according to the invention are cDNAs having a nucleic acid sequence of SEQ ID NO:1, 3, 5, 7, 9, 11, 13, 15, 17, 19, 21, 23, 25, 27, 29, 31, 33, 35, 37, 39, 41, 43 or 45.

Nucleic acids which hybridize under stringent conditions with sequences of SEQ ID NO:1, 3, 5, 7, 9, 11, 13, 15, 17, 19, 21, 23, 25, 27, 29, 31, 33, 35, 37, 39, 41, 43 or 45 are likewise included in the present invention.

The term “to hybridize” as used in the present context describes the process during which a single-stranded nucleic acid molecule undergoes base pairing with a complementary strand. Starting from the sequence information disclosed herein, this allows, for example, DNA fragments to be isolated from insects other than Drosophila melanogaster which encode polypeptides with the biological activity of receptors.

Preferred hybridization conditions are given below:

Hybridization solution: 6×SSC /0% formamide, preferred hybridization solution: 6×SSC/25% formamide.

Hybridization temperature: 34° C., preferred hybridization temperature: 42° C.

Wash step 1: 2×SSC at 40° C.,

Wash step 2: 2×SSC at 45° C.; preferred wash step 2: 0.6×SSC at 55° C.; particularly preferred wash step 2: 0.3×SSC at 65° C.

The present invention furthermore encompasses nucleic acids which have at least 70% identity, preferably at least 80% identity, particularly preferably at least 90% identity, very particularly preferably at least 95% identity, with a sequence of SEQ ID NO:1, 3, 5, 7, 9, 11, 13, 15, 17, 19, 21, 23, 25, 27, 29, 31, 33, 35, 37, 39, 41, 43 or 45 over a length of at least 20, preferably at least 25, particularly preferably at least 30, consecutive nucleotides, and very particularly preferably over their full length.

The degree of identity of the nucleic acid sequences is preferably determined with the aid of the program GAP from the program package GCG, Version 9.1, using standard settings.

The present invention furthermore provides DNA constructs which comprise a nucleic acid according to the invention and a heterologous promoter.

The term “heterologous promoter” as used in the present context refers to a promoter which has properties which differ from the properties of the promoter which controls the expression of the gene in question in the original organism. The term “promoter” as used in the present context generally refers to expression control sequences. The choice of heterologous promoters depends on whether pro- or eukaryotic cells or cell-free systems are used for expression. Examples of heterologous promoters are the early or late promoter of SV40, of the adenovirus or of the cytomegalovirus, the lac system, the trp system, the main operator and promoter regions of the lambda phage, the fd coat protein control regions, the 3-phosphoglycerate kinase promoter, the acid phosphatase promoter and the yeast α-mating factor promoter.

The invention furthermore provides vectors which contain a nucleic acid according to the invention or a DNA construct according to the invention. All plasmids, phasmids, cosmids, YACs or synthetic chromosomes used in molecular biology laboratories can be used as vectors.

The present invention also provides host cells which contain a nucleic acid according to the invention, a DNA construct according to the invention or a vector according to the invention.

The term “host cell” as used in the present context refers to cells which do not naturally comprise the nucleic acids according to the invention.

Suitable host cells are both prokaryotic cells, such as bacteria from the genera Bacillus, Pseudomonas, Streptomyces, Streptococcus, Staphylococcus, preferably E. coli, and eukaryotic cells, such as yeasts, mammalian cells, amphibian cells, insect cells or plant cells. Preferred eukaryotic host cells are HEK-293, Schneider S2, Spodoptera Sf9, Kc, CHO, COS1, COS7, HeLa, C127, 3T3 or BHK cells and, in particular, Xenopus oocytes.

The invention furthermore provides antibodies which bind specifically to the above-mentioned polypeptides or receptors. Such antibodies are produced in the customary manner. For example, such antibodies may be produced by injecting a substantially immunocompetent host with such an amount of a polypeptide according to the invention or a fragment thereof which is effective for antibody production, and subsequently obtaining this antibody. Furthennore, an immortalized cell line which produces monoclonal antibodies may be obtained in a manner known per se. If appropriate, the antibodies may be labelled with a detection reagent. Preferred examples of such a detection reagent are enzymes, radiolabelled elements, fluorescent chemicals or biotin. Instead of the complete antibody, it is also possible to employ fragments which have the desired specific binding properties. The term “antibodies” as used in the present context therefore also extends to parts of complete antibodies, such as Fa, F(ab′)₂ or Fv fragments, which are still capable of binding to the epitopes of the polypeptides according to the invention.

The nucleic acids according to the invention can be used, in particular, for generating transgenic invertebrates. These may be employed in assay systems which are based on an expression, of the polypeptides according to the invention, which deviates from the wild type. Based on the information disclosed herein, it is furthermore possible to generate transgenic invertebrates where expression of the polypeptides according to the invention is altered owing to the modification of other genes or promoters.

The transgenic invertebrates are generated, for example, in the case of Drosophila melanogaster, by P-element-mediated gene transfer (Hay et al., 1997) or, in Caenorhabditis elegans, by transposon-mediated gene transfer (for example by Tcl; Plasterk, 1996).

The invention therefore also provides transgenic invertebrates which contain at least one of the nucleic acids according to the invention, preferably transgenic invertebrates of the species Drosophila melanogaster or Caenorhabditis elegans, and their transgenic progeny. The transgenic invertebrates preferably contain the polypeptides according to the invention in a form which deviates from the wild type.

The present invention furthermore provides methods of producing the polypeptides according to the invention. To produce the polypeptides encoded by the nucleic acids according to the invention, host cells which contain one of the nucleic acids according to the invention can be cultured under suitable conditions, where the nucleic acid to be expressed may be adapted to the codon usage of the host cells. Thereupon, the desired polypeptides can be isolated from the cells or the culture medium in a customary manner. The polypeptides may also be produced in in vitro systems.

A rapid method of isolating the polypeptides according to the invention which are synthesized by host cells using a nucleic acid according to the invention starts with the expression of a fusion protein, it being possible for the fusion partner to be affinity-purified in a simple manner. For example, the fusion partner may be glutathione S-transferase. The fusion protein can then be purified on a glutathione affinity column. The fusion partner can then be removed by partial proteolytic cleavage, for example at linkers between the fusion partner and the polypeptide according to the invention to be purified. The linker can be designed such that it includes target amino acids, such as arginine and lysine residues, which define sites for trypsin cleavage. To generate such linkers, standard cloning methods using oligonucleotides may be employed.

Other purification methods which are possible are based on preparative electrophoresis, FPLC, HPLC (for example using gel filtration, reversed-phase or moderately hydrophobic columns), gel filtration, differential precipitation, ion-exchange chromatography and affinity chromatography.

Since the receptors constitute membrane proteins, the purification methods preferably involve detergent extractions, for example using detergents which have no, or little, effect on the secondary and tertiary structures of the polypeptides, such as nonionic detergents.

The purification of the polypeptides according to the invention can encompass the isolation of membranes, starting from host cells which express the nucleic acids according to the invention. Such cells preferably express the polypeptides according to the invention in a sufficiently high copy number, so that the polypeptide quantity in a membrane fraction is at least 10 times higher than that in comparable membranes of cells which naturally express the receptors; particularly preferably, the quantity is at least 100 times, very particularly preferably at least 1,000 times, higher.

The terms “isolation or purification” as used in the present context mean that the polypeptides according to the invention are separated from other proteins or other macromolecules of the cell or of the tissue. The protein content of a composition containing the polypeptides according to the invention is preferably at least 10 times, particularly preferably at least 100 times, higher than in a host cell preparation.

The polypeptides according to the invention may also be affinity-purified without a fusion partner with the aid of antibodies which bind to the polypeptides.

The present invention furthermore provides methods for producing the nucleic acids according to the invention. The nucleic acids according to the invention can be produced in a customary manner. For example, all of the nucleic acid molecules can be synthesized chemically, or else only short sections of the sequences according to the invention can be synthesized chemically, and such oligonucleotides can be radiolabelled or labelled with a fluorescent dye. The labelled oligonucleotides can be used for screening cDNA libraries generated starting from insect mRNA or for screening genomic libraries generated starting from insect genomic DNA. Clones which hybridize with the labelled oligonucleotides are chosen for isolating the DNA in question. After characterization of the isolated DNA, the nucleic acids according to the invention are obtained in a simple manner.

Alternatively, the nucleic acids according to the invention can also be produced by means of PCR methods using chemically synthesized oligonucleotides.

The term “oligonucleotide(s)” as used in the present context denotes DNA molecules composed of 10 to 50 nucleotides, preferably 15 to 30 nucleotides. They are synthesized chemically and can be used as probes.

The nucleic acids or polypeptides according to the invention allow novel active compounds for crop protection and/or pharmaceutically active compounds for the treatment of humans and animals to be identified, such as chemical compounds which, being modulators, in particular agonists or antagonists, alter the properties of the receptors according to the invention. To this end, a recombinant DNA molecule comprising at least one nucleic acid according to the invention is introduced into a suitable host cell. The host cell is grown in the presence of a compound or a probe comprising a variety of compounds under conditions which allow expression of the receptors according to the invention. A change in the receptor properties can be detected, for example, as described below in Example 2. This allows, for example, insecticidal substances to be found.

Receptors alter the concentration of intracellular cAMP via interaction with G-proteins, preferably after previously having been activated. Thus, changes in the receptor properties by chemical compounds can be measured after heterologous expression, for example by measuring the intracellular cAMP concentrations directly via ELISA assay systems (Biomol, Hamburg, Germany) or RIA assay systems (NEN, Schwalbach, Germany) in HTS format. An indirect measurement of the cAMP concentration is possible with the aid of reporter genes (for example luciferase), whose expression depends on the cAMP concentration (Stratowa et al., 1995). The coexpression of receptors with specific G-proteins, for example Gα15, Gα16 or else chimeric G-proteins, in heterologous systems and measuring the increase in calcium, for example using fluorescent dyes or equorin, is an alternative possibility of carrying out the screening (Stables et al., 1997, Conklin et al., 1993).

Furthermore, the binding of GTP to the activated G-protein can be used as a read-out system for assaying substances. Also, binding experiments with labelled peptides can be employed for screening.

The term “agonist” as used in the present context refers to a molecule which activates the receptor.

The term “antagonist” as used in the present context refers to a molecule which displaces an agonist from its binding site.

The term “modulator” as used in the present context constitutes the generic term for agonist and antagonist. Modulators can be small organochemical molecules, peptides or antibodies which bind to the polypeptides according to the invention. Other modulators may be small organochemical molecules, peptides or antibodies which bind to a molecule which, in turn, binds to the polypeptides according to the invention, thus affecting their biological activity. Modulators may constitute mimetics or natural substrates and ligands.

The modulators are preferably small organochemical compounds.

The binding of the modulators to the polypeptides according to the invention can alter the cellular processes in a manner which leads to the death of the insects treated therewith.

The present invention therefore also extends to the use of modulators of the polypeptides according to the invention as insecticides or pharmaceuticals.

The nucleic acids or polypeptides according to the invention also allow compounds to be found which bind to the receptors according to the invention. Again, these can be used as insecticides on plants or as pharmaceutically active compounds for the treatment of humans and animals. For example, host cells which contain the nucleic acids according to the invention and which express the corresponding receptors or polypeptides, or the gene products themselves, are brought into contact with a compound or a mixture of compounds under conditions which permit the interaction of at least one compound with the host cells, the receptors or the individual polypeptides.

Using host cells or transgenic invertebrates which contain the nucleic acids according to the invention, it is also possible to find substances which alter receptor expression.

The above-described nucleic acids according to the invention, vectors and regulatory regions can furthermore be used for finding genes which encode polypeptides which participate in the synthesis, in insects, of functionally similar receptors. Functionally similar receptors are to be understood as meaning in accordance with the present invention receptors which comprise polypeptides which, while differing from the amino acid sequence of the polypeptides described herein, essentially have the same functions.

### Information on the Sequence Listing and the Figures

SEQ ID NO:1, 3, 5, 7, 9, 11, 13, 15, 17, 19, 21, 23, 25, 27, 29, 31, 33, 35, 37, 39, 41, 43 and 45 show the nucleotide and amino acid sequences of the isolated receptor cDNAs. SEQ ID NO:2, 4, 6, 8, 10, 12, 14, 16, 18, 20, 22, 24, 26, 28, 30, 32, 34, 36, 38, 40, 42, 44 and 46 furthermore show the amino acid sequences of the proteins deduced from the receptor cDNA sequences.

### EXAMPLES

### Example 1

Isolation of the above-described polynucleotides

Polynucleotides were manipulated by standard methods of recombinant DNA technology (Sambrook et al., 1989). Nucleotide and protein sequences were bioinformatically processed using the program package GCG Version 9.1 (GCG Genetics Computer Group, Inc., Madison Wis., USA).

Isolation of poly-A-containing RNA from Drosophila tissue and construction of the cDNA libraries.

The RNA for the cDNA library I was isolated from whole Drosophila melanogaster embryos and larvae (RNAzol, Life Technologies, Karlsruhe, Germany, following the instructions of the manufacturer). From this RNA, the poly-A-containing RNAs were then isolated by purification using Dyna Beads 280 (Dynal, Hamburg, Germany). 5 μg of these poly-A-containing RNAs were then employed for constructing the cDNA library using the λ-ZAP-CMV vector (cDNA Synthesis Kit, ZAP-cDNA Synthesis Kit and ZAP-cDNA Gigapack III Gold Cloning Kit, all from Stratagene-Europe, Amsterdam, the Netherlands).

Generation of plasmid pools

Following the instructions of the manufacturer, the cDNA library in Lambda-pCMV was subjected to mass in-vivo-excision to generate a phagemide library. 10×96 minipreparation cultures were then sown, each preparation calculated to contain 1,000 clones. The DNA was then purified using the Qiawell Ultra DNA preparation system from Qiagen (Hilden, Germany) and deposited in 96-well microtitre plates. In this way, the library was represented in the form of 960 pools of 1,000 cDNA clones each.

PCR with library pools.

Each microtitre plate was copied to a meta pool which represented the entire plate. In each case 0.5 μl of this meta pool was used for a PCR with the following oligodeoxynucleotide primers:

Primer 1s: AAGGTCATCAAAATGCTGATT (SEQ ID NO:47)

Primer 1a: ATTGTAGCAGCTATTGCTCAT (SEQ ID NO:48)

Primer 2s: CAGCTCGTTCGATTCGGTCCT (SEQ ID NO:49)

Primer 2a: GTGACAGCGGTCATAGTCCGA (SEQ ID NO:50)

Primer 3s: ATCGAGGCATCCACCTATGGC (SEQ ID NO:51)

Primer 3a: AGGTGGGCGCAGGCATCGTAG (SEQ ID NO:52)

Primer 4s: TTGCTACGTAGTTCTGAGGAATC (SEQ ID NO:53)

Primer 4a: ATGCAGGTGGAGAGCTTCATG (SEQ ID NO:54)

Primer 5s: TGGCAGACGAGTGCTTCCTGA (SEQ ID NO:55)

Primer 5a: GGACCGCTGAAGTTGACCAG (SEQ ID NO:56)

Primer 6s: TGGTCTGGTACCTGCTGGTCA (SEQ ID NO:57)

Primer 6a: GCGATGAGCCATTTGACCAGC (SEQ ID NO:58)

Primer 7s: GTGACCCATGCGTTCATCATC (SEQ ID NO:59)

Primer 7a: CTGCAGCATGGGCAGAAAG (SEQ ID NO:60)

Primer 8s: GCGATCACCTGGAAGATCTGC (SEQ ID NO:61)

Primer 8a: TGGTGATGCCAATAGGATACC (SEQ ID NO:62)

Primer 9s: CTGTTGCACTTCCTGGTCTAC (SEQ ID NO:63)

Primer 9a: ACGCACAGCTCCCTGAATTTC (SEQ ID NO:64)

Primer 10s: GAGGAGCACGATGTGAGTGG (SEQ ID NO:65)

Primer 10a: CGTGTAAACGGATAATTCTG (SEQ ID NO:66)

Primer 11s: GTCTGGCTGATACCCAGCTA (SEQ ID NO:67)

Primer 11a: ACGCTCTTGACTTTCTCGAAC (SEQ ID NO:68)

Primer 12s: TTCGCACACCAGTTCTACGAC (SEQ ID NO:69)

Primer 12a: GCGTTCATGAAGCAGTAGGTG (SEQ ID NO:70)

Primer 13s: TACATCTGCATCGGACGTGG (SEQ ID NO:71)

Primer 13a: CACTATGCCGCATTGCTCC (SEQ ID NO:72)

Primer 14s: GTGATCTATGTGGTGATGAGG (SEQ ID NO:73)

Primer 14a: CCTCGACTGCACGGTGCTGGC (SEQ ID NO:74)

Primer 15s: GTCATCGTTCTGGGCAATTCA (SEQ ID NO:75)

Primer 15a: GATGCTCATGGCCACCAGCAC (SEQ ID NO:76)

Primer 16s: AACGTACTGCGAGTGATCGTG (SEQ ID NO:77)

Primer 16a: AATGGCAAAGGTGACATCGTG (SEQ ID NO:78)

Primer 17s: ATGTGCCGCATCAGCGAGTTC (SEQ ID NO:79)

Primer 17a: GTTACCGGTGGCTGTGAACAC (SEQ ID NO:80)

Primer 18s: CAGAAACCGCTCAAGGAGACG (SEQ ID NO:81)

Primer 18a: CCTCAGACGAGCCGCAGTTAG (SEQ ID NO:82)

Primer 19s: GCACTGGCACTGCTGCTGG (SEQ ID NO:83)

Primer 19a: CACAGCCACCACGGTGATGC (SEQ ID NO:84)

Primer 20s: GGCACTTTGCCGTGGATAGTG (SEQ ID NO:85)

Primer 20a: GAACCGATCGATGGACATCAG (SEQ ID NO:86)

Primer 21s: CATCAGCTCCTACCTGCTGC (SEQ ID NO:87)

Primer 21a: CGTAGAGCAGCGGATTGATAC (SEQ ID NO:88)

Primer 22s: CATCTCACTGGCCTGCAGTGA (SEQ ID NO:89)

Primer 22a: CATGCTCAGAGTCGACTTCG (SEQ ID NO:90)

Primer 23s: GCGGTAATGGCACTGTTCTC (SEQ ID NO:91)

Primer 23a: GGATACTGTGGAGAACCGGTA (SEQ ID NO:92)

The PCR parameters were as follows: 94° C., 1 min; 35 times (94° C., 30 s; 55° C., 30 s; 72° C., 45 s). The PCRs were carried out on a Biometra Uno II (Biometra, Göttingen, Germany).

Library pools which were positive in the PCR were transformed in X1-1 Blue (Stratagene, Amsterdam, the Netherlands) and subjected to a colony lift (Sambrook et al., 1989). The probe used for the hybridization was a PCR product of the reaction with the respective primer pair (hybridization and detection by means of BrightStar, psoralene-biotin kit, Ambion, Austin, Tex., USA), labelled using psoralene-biotin (BrightStar, psoralene-biotin kit, Ambion, Austin, Tex., USA). Positive colonies were selected and grown, and the DNA was isolated by plasmid preparation (Qiagen, Hilden, Germany).

For identification, the isolated gene library plasmids were subjected to incipient sequencing (ABI Prism Dye Terminator Cycle Sequencing Kit, ABI, using the ABI prism 310 genetic analyser, ABI-Deutschland, Weiterstadt, Germany) using T3 and T7 primers. The complete polynucleotide sequences of the DB3 were determined by primer walking by means of the Cycle Sequencing ABI Prism Dye Terminator Cycle Sequencing Kit, ABI, using an ABI prism 310 genetic analyser (ABI-Deutschland, Weiterstadt, Germany).

### Example 2

The sequences of SEQ ID NO:2, 4, 6, 8, 10, 12, 14, 16, 18, 20, 22, 24, 26, 28, 30, 32, 34, 36, 38, 40, 42, 44 or 46 were assigned by blast analysis (Altschul et al., 1997). What is shown is in each case the best hit from the blast analysis (Swissprot database of 4 March 2000). The E-value parameter is a measure for the non-randomness of the assignment. With sufficient reliability, all sequences were identified as neuropeptide receptors.

### Sequence Comparison and Assignment of the Sequences

| | | Accession No./Accession from Swissprot database |
|---|---|---|
| Seq ID | E value | (4 March 2000) |
| 2 | 8e-45 | P25931/NEUROPEPTIDE Y RECEPTOR (NPY-R) |
| | | (PR4 RECEPTOR) |
| 4 | 1e-81 | Q16983/DIURETIC HORMONE RECEPTOR |
| | | PRECURSOR (DH-R) |
| 6 | 1e-76 | Q16983/DIURETIC HORMONE RECEPTOR |
| | | PRECURSOR (DH-R) |
| 8 | 7e-41 | Q9Z2D5/NEUROPEPTIDE Y RECEPTOR TYPE 2 |
| | | (NPY2-R) |
| 10 | 8e-52 | P35346/SOMATOSTATIN RECEPTOR TYPE 5 |
| | | (SS5R) |
| 12 | | Q16602/CALCITONIN GENE-RELATED PEPTIDE |
| | | TYPE 1 RECEPTOR |
| 14 | 2e-05 | P56718/OREXIN RECEPTOR TYPE 1 (OX1R) |
| 16 | 6e-25 | Q9Z2D5/NEUROPEPTIDE Y RECEPTOR TYPE 2 |
| | | (NPY2-R) |
| 18 | 1e-40 | P21729/GASTRIN-RELEASING PEPTIDE |
| | | RECEPTOR (GRP-R) |
| 20 | 1e-86 | O02721/LUTROPIN-CHORIOGONADOTROPIC |
| | | HORMONE RECEPTOR |
| 22 | 6e-19 | Q63931/CHOLECYSTOKININ TYPE A |
| | | RECEPTOR (CCK-A RECEPTOR) |
| 24 | 1e-33 | P56418/GASTRIN/CHOLECYSTOKININ TYPE B |
| | | RECEPTOR |
| 26 | 4e-34 | Q95254/GROWTH HORMONE SECRETAGOGUE |
| | | RECEPTOR TYPE 1 |
| 28 | 7e-54 | P34993/SOMATOSTATIN RECEPTOR TYPE 2 |
| | | (SS2R) |
| 30 | 3e-41 | P48043/VASOPRESSIN V1A RECEPTOR |
| 32 | 5e-42 | Q60755/CALCITONIN RECEPTOR PRECURSOR |
| | | (CT-R) |
| 34 | 7e-39 | P47751/PHE(13) BOMBESIN RECEPTOR |
| 36 | 6e-22 | P70031/CHOLECYSTOKININ RECEPTOR |
| | | (CCK-XLR) |
| 38 | 6e-41 | Q9Z2D5/NEUROPEPTIDE Y RECEPTOR TYPE 2 |
| | | (NPY2-R) |
| 40 | 1e-19 | O88626/GALANIN RECEPTOR TYPE 3 (GAL3-R) |
| 42 | 5e-15 | P47211/GALANIN RECEPTOR TYPE 1 (GAL1-R) |
| 44 | 2e-08 | Q95254/GROWTH HORMONE SECRETAGOGUE |
| | | RECEPTOR TYPE 1 |
| 46 | 7e-17 | Q62463/VASOPRESSIN V1A RECEPTOR |

### Example 3

### Heterologous Expression

The receptors from insects can be expressed functionally in xenopus ooctyes. To this end, G-protein-activatable potassium channels (GIRK1 and GIRK4) are coexpressed in order to measure activation of the receptors (White et al., 1998). The nucleotide sequences according to the invention were used directly for the expression experiments, since they were already in an expression vector with CMV promoter.

### Oocyte Measurements

### 1. Oocyte Preparation

The oocytes are obtained from an adult female Xenopus laevis frog (Horst Kähler, Hamburg, Germany). The frogs are kept in large tanks with circulating water at a water temperature of 20-24° C. Parts of the frog ovary are removed through a small incision in the abdomen (approx. 1 cm), with full anaesthesia. The ovary is then treated for approximately 140 min with 25 ml of collagenase (type I, C-0130, SIGMA-ALDRICH CHEMIE GmbH, Deisenhofen, Germany; 355 U/ml, prepared with Barth's solution without calcium in mM: NaCl 88, KCl 1, MgSO₄ 0.82, Na—HCO₃ 2.4, Tris/HCl 5, pH 7.4), with constant shaking. Then, the oocytes are washed with Barth's solution without calcium. Only oocytes at maturity stage V (Dumont, 1972) are selected for the further treatment and transferred into microtitre plates (Nunc MicroWell™ plates, Cat. No. 245128+263339 (lid), Nunc GmbH & Co. KG, Wiesbaden, Germany), filled with Barth's solution (in mM: NaCl 88, KCl 1, MgSO₄ 0.82, Ca(NO₃)₂ 0.33, CaCl₂ 0.41, NaHCO₃ 2.4, Tris/HCl 5, pH 7.4) and gentamicin (gentamicin sulphate, G-3632, SIGMA-ALDRICH CHEMIE GmbH, Deisenhofen, Germany; 100 U/ml). The oocytes are then kept in a cooling incubator (type KB 53, WTB Binder Labortechnik GmbH, Tuttlingen, Germany) at 19.2° C.

### 2. Injecting the Oocytes

Injection electrodes of diameter 10-15 μm are prepared using a pipette-drawing device (type L/M-3P-A, list-electronic, Darmstadt-Eberstadt, Germany). Prior to injection, aliquots with the receptor DNA or GIRK1/4-DNA are defrosted and diluted with water to a final concentration of 10 ng/μl. The DNA samples are centrifuged for 120 s at 3 200 g (type Biofuge 13, Heraeus Instruments GmbH, Hanau, Germany). An extended PE tube is subsequently used as transfer tube to fill the pipettes from the rear end. The injection electrodes are attached to a X,Y,Z positioning system (treatment center EP1090, isel-automation, Eiterfeld, Germany). With the aid of a Macintosh Computer, the oocytes in the microtitre plate wells are approached, and approximately 50 nl of the DNA solution are injected into the oocytes by briefly applying a pressure (0.5-3.0 bar, 3-6 s).

### 3. Electrophysiological Measurements

A two-electrode voltage clamp equipped with a TURBO TEC-10CD (npi electronic GmbH, Tamm, Germany) amplifier is used to carry out the electrophysiological measurements. The micropipettes required for this purpose are drawn in two movements from aluminium silicate glass (capillary tube, Art. No. 14 630 29, 1=100 mm, Ø_{ext.}=1.60 mm, Ø_{int.}=1.22 mm, Hilgenberg GmbH, Malsfeld, Germany) (Hamill et al., 1981). Current and voltage electrodes have a diameter of 1-3 μm and are filled with 1.5 M KCl and 1.5 M potassium acetate. The pipettes have a capacitance of 0.2-0.5 MW. To carry out the electrophysiological measurements, the oocytes are transferred into a small chamber which is flushed continuously with normal Rimland solution (in mM: KCl 90, MgCl₂ 3, HEPES 5, pH 7.2). To apply a substance, the perfusion solution is exchanged for a substance solution of the same composition and additionally the desired substance concentration. The successful expression of the receptor DNA is checked after one week at a clamp potential of −60 mV. Unresponsive oocytes are discarded. All the others are used for substance testing. The data are documented by means of a YT plotter (YT plotter, model BD 111, Kipp & Zonen Delft BV, AM Delft, the Netherlands). When test substances are assayed in concentration series, these measurements are carried out on at least two different oocytes and at at least five different concentrations. The substances are assayed directly with-out preincubation in the presence of glutamate (gamma-amino-N-butyric acid, A2129, SIGMA-ALDRICH CHEMIE GmbH, Deisenhofen, Germany) for their antagonists. The individual data are entered in Origin (evaluation software Microcal Origin, Microcal Software, Inc., Northampton, Mass. 01060-4410 USA [lacuna] (Additive GmbH, Friedrichsdorf/Ts, Germany). Means, standard deviation, IC₅₀ values and IC₅₀ curves are calculated using Origin. These measurements are carried out at least in duplicate.

### References

Altschul et al. (1997), Gapped BLAST and PSI-BLAST: a new generation of protein database search programs, Nucleic Acids Res. 25:3389-3402.

Birgul, N. et al. (1999), Reverse physiology in Drosophila: identification of a novel allatostatin-like neuropeptide and its cognate receptor structurally related to the mammalian somatostatin/galanin/opioid receptor family, EMBO Journal 18, 5892-5900.

Brown, B. E. und Starrall, A. N. (1975), Isolation of proctolin, a myotropic peptide from Periplaneta americana, Journal of Insect Physiology 21, 1879-1881.

Coast, G. M. (1998), Insect Diuretic Peptides: Structures, Evolution and Actions, American Zoology 38, 442-449.

Conklin et al. (1993), Substitution of three amino acids switches receptor specificity of Gq alpha to that of Gi alpha, Nature 20;363(6426):274-6.

Devereux et al. (1984), Nucleic Acids Research 12, 387.

Dumont, J. N. (1972), Oogenesis in Xenopus laevis (Daudin). 1. Stages of oocyte development in laboratory maintained animals, J. Morphol. 136: 153-180.

Gäde , G. (1997a), The explosion of structural information on insect neuropeptides, In: Progress in the chemistry of organic natural products (Herz, W., Kirby, G. W., Moore, R. E., Steglich, W., Tamm, C. eds), 1-128.

Gäde , G. et al. (1997b), Hormonal regulation in Insects: Facts, Gaps, and Future Directions, Physiological Reviews 77, 963-1032.

Hamill, O. P., Marty, A., Neher, E., Sakmann, B. Sigworth, F. J. (1981), Improved patch-clamp techniques for high-resolution current recording from cells and cell-free membrane patches, Pfügers Arch. 391: 85-100.

Hay et al. (1997), P element insertion-dependent gene activation in the Drosophila eye, Proceedings of The National Academy of Sciences of The United States of America 94 (10), 5195-5200.

Holman, G. M. et al. (1986), Isolation, primary structure and synthesis of two neuropeptides from Leucophaea maderae: Members of a new family of Cephalomaotrophins, Comparative Biochemical Physiology 84C, 205.

Holman, G. M. (1991), Insect myotropic peptides: isolation, structural characterization and biological properties, In: Insect Neuropeptides: Chemistry, Biology and Action (Menn, J. J., Kelly, T. J., Masler, E. P., eds), 40-50.

King, F. D. und Wilson, S. (1999), Recent advances in 7-transmembrane receptor research, Current Opinion in Drug Discovery & Development 2, 83-95.

Lagueux, M. et al. (1990), cDNAs from neurosecretory cells of brains of Locusta migratoria encoding a novel member of the superfamily of insulins, European Journal of Biochemistry 187, 249-254.

Osborne, R. H. (1996), Insect Neurotransmission: Neurotransmitters and their Receptors, Pharmacology & Therapeutics 69, 117-142.

Plasterk (1996), The Tcl/mariner transposon family, Transposable Elements/Current Topics in Microbiology and Immunology 204, 125-143

Ramsey, J. A. (1954), Active transport of water by the Malpighian tubules of the stick insect, Dixippus morosus, Journal of Experimental Biology 31, 104-113.

Reagan, J. D. (1994), Expression cloning of an insect diuretic hormone receptor, Journal of Biological Chemistry 269, 9-12.

Sambrook et al. (1989), Molecular Cloning, A Laboratory Manual, 2nd ed. Cold Spring Harbor Press.

Stables et al. (1997), A Bioluminescent Assay for Agonist Activity at Potentially Any G-protein coupled Receptor, Analytical Biochemistry 252, 115-126.

Stratowa C et al. (1995), Use of a luciferase reporter system for characterizing G-protein-linked receptors, Current Opinion in Biotechnology 6, 574-581.

Tobe, S. S. et al. (1994), Allatostatins, peptide inhibitors of juvenile hormone production in insects, In: Perspectives in Comparative Endocrinology (Davey, K. G., Peter, R. E., Tobe, S. S., eds), 12-19.

Vanden Broeck, J. et al. (1997), Insect Neuropeptides and Their Receptors, Trends in Endocrinology & Metabolism 8, 321-326.

White J H et al. (1998), Heterodimerization is required for the formation of a functional GABA(B) receptor, Nature 396, 679-82.

1. Polypeptide having the biological activity of a peptide receptor and comprising an amino acid sequence which has at least 70% identity with a sequence of SEQ ID NO:2, 4, 6, 8, 10, 12, 14, 16, 18, 20, 22, 24, 26, 28, 30, 32, 34, 36, 38, 40, 42, 44 or 46.

2. Polypeptide according to claim 1, characterized in that the amino acid sequence corresponds to a sequence of SEQ ID NO:2, 4, 6, 8, 10, 12, 14, 16, 18, 20, 22, 24, 26, 28, 30, 32, 34, 36, 38, 40, 42, 44 or 46.

3. Nucleic acid comprising a nucleotide sequence which encodes a polypeptide according to claim 1 or 2.

4. Nucleic acid according to claim 3, characterized in that it is single- or double-stranded DNA or RNA.

5. Nucleic acid according to claim 4, characterized in that it is a fragment of genomic DNA or cDNA.

6. Nucleic acid according to claim 3, characterized in that the nucleotide sequence corresponds to a sequence of SEQ ID NO:1, 3, 5, 7, 9, 11, 13, 15, 17, 19, 21, 23, 25, 27, 29, 31, 33, 35, 37, 39, 41, 43 or 45.

7. Nucleic acid according to claim 3, characterized in that it hybridizes under stringent conditions to the sequences of SEQ ID NO:1, 3, 5, 7, 9, 11, 13, 15, 17, 19, 21, 23, 25, 27, 29, 31, 33, 35, 37, 39, 41, 43 or 45.

8. DNA construct comprising a nucleic acid according to any of claims 3 to 7 and a heterologous promoter.

9. Vector comprising a nucleic acid according to any of claims 3 to 7 or a DNA construct according to claim 8.

10. Vector according to claim 9, characterized in that the nucleic acid is operatively linked to regulatory sequences which ensure the expression of the nucleic acid in pro- or eukaryotic cells.

11. Host cell containing a nucleic acid according to any of claims 3 to 7, a DNA construct according to claim 8 or a vector according to claim 9 or 10.

12. Host cell according to claim 11, characterized in that it is a prokaryotic cell, in particular E. coli.

13. Host cell according to claim 11, characterized in that it is a eukaryotic cell, in particular a mammalian or insect cell.

14. Antibody which binds specifically to a polypeptide according to claim 1 or 2.

15. Transgenic invertebrate containing a nucleic acid according to any of claims 3 to 7.

16. Transgenic invertebrate according to claim 15, characterized in that it is Drosophila melanogaster or Caenorhabditis elegans.

17. Transgenic progeny of an invertebrate according to claim 15 or 16.

18. Method of producing a polypeptide according to claim 1 or 2, comprising
(a) culturing a host cell according to any of claims 11 to 13 under conditions which ensure the expression of the nucleic acid according to any of claims 3 to 7, or
(b) expressing a nucleic acid according to any of claims 3 to 7 in an in vitro system, and
(c) obtaining the polypeptide from the cell, the culture medium or the in vitro system.

19. Method of producing a nucleic acid according to any of claims 3 to 7, comprising the following steps:
(a) full chemical synthesis in a manner known per se, or
(b) chemical synthesis of oligonucleotides, labelling of the oligonucleotides, hybridizing the oligonucleotides to DNA of a genomic library or cDNA library generated from insect genomic DNA or insect MRNA, respectively, selecting positive clones and isolating the hybridizing DNA from positive clones, or
(c) chemical synthesis of oligonucleotides and amplificiation of the target DNA by means of PCR.

20. Method of producing a transgenic invertebrate according to claim 15 or 16, which comprises introducing a nucleic acid according to any of claims 3 to 7 or a vector according to claim 9 or 10.

21. Method of finding novel active compounds for crop protection, in particular compounds which alter the properties of polypeptides according to claim 1 or 2, comprising the following steps:
(a) providing a host cell according to any of claims 11 to 13,
(b) culturing the host cell in the presence of a chemical compound or a mixture of chemical compounds, and
(c) detecting altered properties.

22. Method of finding a chemical compound which binds to a polypeptide according to claim 1 or 2, comprising the following steps:
(a) bringing a polypeptide according to claim 1 or 2 or a host cell according to any of claims 11 to 13 into contact with a chemical compound or a mixture of chemical compounds under conditions which permit the interaction of a chemical compound with the polypeptide, and
(b) determining the chemical compound which binds specifically to the polypeptide.

23. Method of finding a chemical compound which alters the expression of a polypeptide according to claim 1 or 2, comprising the following steps:
(a) bringing a host cell according to any of claims 11 to 13 or a transgenic invertebrate according to claim 15 or 16 into contact with a chemical compound or a mixture of chemical compounds,
(b) determining the concentration of the polypeptide according to claim 1 or 2, and
(c) determining the chemical compound which specifically affects the expression of the polypeptide.

24. Use of a polypeptide according to claim 1 or 2, of a nucleic acid according to any of claims 3 to 7, of a vector according to claim 9 or 10, of a host cell according to any of claims 11 to 13, of an antibody according to claim 14 or of a transgenic invertebrate according to claim 15 or 16 for finding novel active compounds for crop protection or finding genes which encode polypeptides which participate in the synthesis of functionally similar peptide receptors in insects.

25. Use of a modulator of a polypeptide according to claim 1 or 2 as insecticide.


SEQUENCE LISTING (92 sequences)

SEQ ID NO: 1 - DNA, 1623 bp, Drosophila melanogaster
  CDS (1)..(1620)
atggacttaatcgagcaggagtcccgcctggaattcctgcccggagccgaggaggaagcagaatttgagcgtctatacgcggctcccgctgagattgtggccctgttgtccattttctatgggggaatcagtatcgtggccgtcattggcaacactttggtcatctgggtggtggccacgaccaggcaaatgcggaccgtgacaaatatgtatatcgctaatttggcttttgccgatgtgattattggcctcttctgcataccatttcagttccaggctgccctgctgcagagttggaacctgccgtggttcatgtgcagcttctgccccttcgtccaggccctgagtgtaaatgtctcggtattcacgctgaccgccattgcaatcgatcggcatagggccatcattaatccacttagggcacgtcccaccaagttcgtatcgaagttcataattggtggaatttggatgctggccctgctatttgcggtgccctttgccattgcctttcgtgtggaggagttgaccgaaagatttcgcgagaacaatgagacctacaatgtgacgcggccattctgcatgaacaagaacctatccgatgatcaattgcaatcctttcgctacaccctggtttttgtgcagtatctggttccattctgtgtcatcagctttgtctacatccagatggcggtacgattgtggggcacacgtgctcctggtaacgcacaggattcacgggacataacgctgttgaaaaacaagaagaaggtcatcaaaatgctgattatcgtggtcattatctttggactctgctggctgccactgcagctctataatattctgtatgtcacgataccggaaatcaacgactaccacttcattagcatcgtctggttttgctgcgattggctggccatgagcaatagctgctacaatccctttatttatggcatctacaatgaaaaatttaagcgggaattcaacaagcgatttgcggcctgtttctgcaagttcaagacgagcatggacgcccacgaaaggaccttttcgatgcacacccgcgccagctccataaggtcaacctacgccaactcctcgatgcgaatccggagtaatctctttggtccggcgcgtggtggtgtcaacaatgggaagccgggcttgcatatgccgcgggtgcatggatccggtgctaacagcggcatttacaacggaagtagtgggcagaacaacaatgtcaatggccaacatcatcagcatcaaagcgtggttacctttgcggccactccgggtgtttcggcaccaggtgttggcgttgcaatgccgccgtggcggcgaaacaacttcaaacctctgcatccgaacgtaatcgaatgcgaggacgacgtggcactcatggagctgccatcaaccacgccccccagcgaggagttggcatccggggccggagtccagttggccctgctaagcagggagagctccagctgcatttgcgaacaggaatttggcagccaaaccgaatgcgatggcacctgcatactcagcgaggtgtcgcgagtccacctgcccggctcgcaggcgaaggacaaggatgcgggcaagtccttgtggcaaccactttaa

SEQ ID NO: 2 - protein, 540 aa, Drosophila melanogaster
MDLIEQESRLEFLPGAEEEAEFERLYAAPAEIVALLSIFYGGISIVAVIGNTLVIWVVATTRQMRTVTNMYIANLAFADVIIGLFCIPFQFQAALLQSWNLPWFMCSFCPFVQALSVNVSVFTLTAIAIDRHRAIINPLRARPTKFVSKFIIGGIWMLALLFAVPFAIAFRVEELTERFRENNETYNVTRPFCMNKNLSDDQLQSFRYTLVFVQYLVPFCVISFVYIQMAVRLWGTRAPGNAQDSRDITLLKNKKKVIKMLIIVVIIFGLCWLPLQLYNILYVTIPEINDYHFISIVWFCCDWLAMSNSCYNPFIYGIYNEKFKREFNKRFAACFCKFKTSMDAHERTFSMHTRASSIRSTYANSSMRIRSNLFGPARGGVNNGKPGLHMPRVHGSGANSGIYNGSSGQNNNVNGQHHQHQSVVTFAATPGVSAPGVGVAMPPWRRNNFKPLHPNVIECEDDVALMELPSTTPPSEELASGAGVQLALLSRESSSCICEQEFGSQTECDGTCILSEVSRVHLPGSQAKDKDAGKSLWQPL

SEQ ID NO: 3 - DNA, 1050 bp, Drosophila melanogaster
  CDS (1)..(1047)
  n 1050: n is a or g or c or t/u, unknown, or other
atgggttttgggctaggtcagttgcgtttacagaggggaaacacgaattatactgcagtaaatgctcaacgaagtttaccttgccaatcattgtatattctcaattgttatttcttacagattttacaatgtcccagctcgttcgattcggtcctgtgctggccacgcacaaacgccggcagtctggctgtactaccctgtttcgaggaattcaagggcgtgcactacgacaccacagacaatgccacccgcttttgctttccaaacggaacgtgggatcactattcggactatgaccgctgtcaccagaactcgggctccataccggtggtgcccgacttctcacccaacgtcgaactgccggccatcatatatgccggcggttatttcctgagcttcgccaccttggtggtggctctcatcatattcctcagctttaaagatcttcgttgcctgcgaaacaccattcatgccaatttgttcctcacctacatcacatccgcactcctctggatactcacactgttcctgcaagtgtacttttacctaaccaactttttctggatgtttgtggagggcctctatctgtacacgctggtggtgcaaacattctccagtgataacattagctttattatctacgccctcatcggctggggctgtccagccgtatgcattttggtgtggtccattgccaaggcatttgctccgcatctcgagaacgagcacttcaatggggtactcatcactaaattacgttctgctcataccctggaaacgcggcagtattacaaggcctcgaaggcgctgctggtgctgatacctctctttggcatcacctatctgttggtgctaacaggcccggaacagggtatcagtcgtaatctcttcgaggccataagagcctttctcataagcacgcagggcttctttgtggctctgttctactgtttccttaactcagaggtgcgccagacgctgaggcatggattcacccggtggcgggagagcaggaatatccatcggaacagttccatcaagaatcgcagn

SEQ ID NO: 4 - protein, 349 aa, Drosophila melanogaster
MGFGLGQLRLQRGNTNYTAVNAQRSLPCQSLYILNCYFLQILQCPSSFDSVLCWPRTNAGSLAVLPCFEEFKGVHYDTTDNATRFCFPNGTWDHYSDYDRCHQNSGSIPVVPDFSPNVELPAIIYAGGYFLSFATLVVALIIFLSFKDLRCLRNTIHANLFLTYITSALLWILTLFLQVYFYLTNFFWMFVEGLYLYTLVVQTFSSDNISFIIYALIGWGCPAVCILVWSIAKAFAPHLENEHFNGVLITKLRSAHTLETRQYYKASKALLVLIPLFGITYLLVLTGPEQGISRNLFEAIRAFLISTQGFFVALFYCFLNSEVRQTLRHGFTRWRESRNIHRNSSIKNR

SEQ ID NO: 5 - DNA, 1113 bp, Drosophila melanogaster
  CDS (1)..(1110)
  n 1113: n is a or g or c or t/u, unknown, or other
atgagtgaccacaaccacatcgattcggtgaacgccagcgggagtgatccgctcctggatctgcacaacctagatggcatcggtgagagcgtggagttgcagtgcctggtgcaggagcacatcgaggcatccacctatggcaacgatagtggccactgcctcacccagttcgactcgatcctgtgctggccgaggacagctcgcggtaccctggcggtgctccagtgcatggacgagttgcagggaattcattacgatagcagcaagaatgcaacgaggttctgccacgccaatggaacttgggagaagtacaccaactacgatgcctgcgcccacctgcccgccccagaatccgtgccggagttcgaggtcatcgtggagctgcccaccattatctactacattggatacaccctcagcctggtatcgctctcgctggcgctgattgttttcgcctacttcaaggagctgcgttgcctgcgcaacaccatccacgccaacttgttcttcacgtacatcatgtcggctttgttctggatactcttgttatccgtccagatctccatccgaagtggagtgggcagctgcattgctctgatcaccctgttccacttcttcaccctgaccaacttcttttggatgctggtcgagggtttgtacctgtacatgctggtggttaagactttctccggggacaatttacgctttaatatctacgcctccataggctggggtggcccggccttgtttgttgtcacctgggctgtggccaagagtctgacggtcacctacagcacccccgaaaaggttctaatcacaaagctgcgctcggccaatacagtggagactcgtcagtataggaaagctgctaaggcactgctggtgctaatcccactttttggcatcacctacctggtggtgctggctggtccctcggaatctggcctaatgggacacatgtttgccgtcctgcgagcggtattactcagcactcagggattctcggtgtcgctgttctactgtttcctcaactcggaggtgcgaaatgcactgagacaccacatttccacgtggcgggacacgcgcaccattcagcttaaccagaaccgacgn

SEQ ID NO: 6 - protein, 370 aa, Drosophila melanogaster
MSDHNHIDSVNASGSDPLLDLHNLDGIGESVELQCLVQEHIEASTYGNDSGHCLTQFDSILCWPRTARGTLAVLQCMDELQGIHYDSSKNATRFCHANGTWEKYTNYDACAHLPAPESVPEFEVIVELPTIIYYIGYTLSLVSLSLALIVFAYFKELRCLRNTIHANLFFTYIMSALFWILLLSVQISIRSGVGSCIALITLFHFFTLTNFFWMLVEGLYLYMLVVKTFSGDNLRFNIYASIGWGGPALFVVTWAVAKSLTVTYSTPEKVLITKLRSANTVETRQYRKAAKALLVLIPLFGITYLVVLAGPSESGLMGHMFAVLRAVLLSTQGFSVSLFYCFLNSEVRNALRHHISTWRDTRTIQLNQNR

SEQ ID NO: 7 - DNA, 1557 bp, Drosophila melanogaster
  CDS (1)..(1554)
atggccaacttaagctggctgagcaccatcaccaccacctcctcctccatcagcaccagccagctgccattggtcagcacaaccaactggagcctaacgtcgccgggaactactagcgctatcttggcggatgtggctgcatcggatgaggataggagcggcgggatcattcacaaccagttcgtgcaaatcttcttctacgtcctgtacgccacggtctttgtcctgggtgtcttcggaaatgtcctggtttgctacgtagttctgaggaatcgggccatgcagactgtgaccaatatattcatcacgaatctggccctgtcggacatattgctctgcgtcctggcggtgccatttactccgctttacacgttcatgggtcgctgggccttcggcaggagtctgtgccatctggtgtcctttgcccagggatgcagcatctacatatccacgctgaccctcacctcgattgccatcgatcggtacttcgttatcatataccccttccatccgcgcatgaagctctccacctgcatcgggatcatagtgagcatctgggtgatagccctgctggccaccgttccctacggcatgtacatgaagatgaccaacgagctggtgaacggaacgcagacaggcaacgagaccctggtggaggccactctaatgctaaacggaagctttgtggcccagggatcaggattcatcgaggcgccggactctacctcggccacccaggcctatatgcaggtgatgaccgccggatcaacgggaccggagatgccctatgtgcgggtgtactgcgaggagaactggccatcggagcagtaccggaaggtgttcggtgccatcacaaccactctgcagtttgtgctgcccttcttcatcatctcgatttgctacgtgtggatatcggtgaagctaaaccagcgggccagggccaagccgggatcgaaatcctcgagacgggaggaggcggatcgggatcgcaagaagcgcaccaaccgcatgctcatcgccatggtggcggtattcggactcagctggctgcccatcaatgtggtcaacatattcgatgacttcgatgacaagtccaacgagtggcgcttctacatcctattcttctttgtggcccactctattgccatgagctccacctgctacaatcccttcctgtacgcctggctgaacgagaacttccgcaaggagttcaagcacgtgctgccctgctttaatccctcgaacaacaacatcatcaacatcaccaggggctataatcggagtgatcggaacacctgtggtccgcgactgcatcatggcaagggggatggtggcatgggcggtggcagtctggacgccgacgaccaggacgagaacggcatcacccaggagacctgtctgcccaaggagaagctgctgattatccccagggagccgacttacggcaatggcacgggtgccgtgtcgccaatccttagcgggcgcggcattaacgccgccctggtgcacggtggcgaccatcagatgcaccagctgcagccgtcacaccatcaacag

SEQ ID NO: 8 - protein, 518 aa, Drosophila melanogaster
MANLSWLSTITTTSSSISTSQLPLVSTTNWSLTSPGTTSAILADVAASDEDRSGGIIHNQFVQIFFYVLYATVFVLGVFGNVLVCYVVLRNRAMQTVTNIFITNLALSDILLCVLAVPFTPLYTFMGRWAFGRSLCHLVSFAQGCSIYISTLTLTSIAIDRYFVIIYPFHPRMKLSTCIGIIVSIWVIALLATVPYGMYMKMTNELVNGTQTGNETLVEATLMLNGSFVAQGSGFIEAPDSTSATQAYMQVMTAGSTGPEMPYVRVYCEENWPSEQYRKVFGAITTTLQFVLPFFIISICYVWISVKLNQRARAKPGSKSSRREEADRDRKKRTNRMLIAMVAVFGLSWLPINVVNIFDDFDDKSNEWRFYILFFFVAHSIAMSSTCYNPFLYAWLNENFRKEFKHVLPCFNPSNNNIINITRGYNRSDRNTCGPRLHHGKGDGGMGGGSLDADDQDENGITQETCLPKEKLLIIPREPTYGNGTGAVSPILSGRGINAALVHGGDHQMHQLQPSHHQ

SEQ ID NO: 9 - DNA, 1404 bp, Drosophila melanogaster
  CDS (1)..(1401)
atgtttacgtggctgatgatggatgtcctccagtttgtgaaaggggaaatgacagccgattcagaggcaaatgccacaaattggtataacacgaacgagagcttatataccacggaactgaaccatagatggattagtggtagttccacaattcagccagaggagtccctttatggcactgatttgcccacctatcaacattgcatagccacgcggaattcctttgctgacttgttcactgtggtgctctacggatttgtgtgcattatcggattatttggcaacaccctggtgatctacgtggtgttgcgcttttccaaaatgcaaacggtcacgaatatatatatcctgaatctggcggtggcagacgagtgcttcctgattggaataccctttctgctgtacacaatgcgaatttgcagctggcgattcggggagtttatgtgcaaagcctacatggtgagcacatccatcacctccttcacctcgtcgatttttctgctcatcatgtccgcggatcgatatatagcggtatgccacccgatttcctcgccacgatatcgaactctgcatattgccaaagtggtctcagcgattgcctggtcaacttcagcggtcctcatgctgcccgtgatcctttatgccagcactgtggagcaggaggatggcatcaattactcgtgcaacataatgtggccagatgcgtacaagaagcattcgggcaccaccttcatactgtacacatttttcctaggattcgccacaccgctgtgctttatcctgagtttctactacttggttataaggaaactgcgatcggtgggtcccaaaccaggaacgaagtccaaggagaagaggcgggctcacaggaaggtcactcgactggtactgacggtggccctgattcactcgaatcccgcgcaaagggacctctcccgactggaaatactcattttcctacttctgggggcactggtttactcgaattcggcggtgaatcccatactttatgccttcctaagtgagaacttccggaagagcttcttcaaggcctttacctgtatgaataagcaggatatcaacgctcaactccagctggagcccagtgttttcaccaaacagggcagtaaaaagaggggtggctccaagcgcctgttgaccagcaatccgcagattcctccactgctgccactgaatgcgggtaacaacaattcatcgaccaccacatcctcgaccacgacagcggaaaagaccggaaccacggggacacagaaatcatgcaattccaatggcaaagtgacagctccgccggagaatttgattatatgtttgagcgagcagcaggaggcattttgcaccaccgcgaggagaggatcgggcgcagtgcagcagacagatttgtaa

SEQ ID NO: 10 - protein, 467 aa, Drosophila melanogaster
MFTWLMMDVLQFVKGEMTADSEANATNWYNTNESLYTTELNHRWISGSSTIQPEESLYGTDLPTYQHCIATRNSFADLFTVVLYGFVCIIGLFGNTLVIYVVLRFSKMQTVTNIYILNLAVADECFLIGIPFLLYTMRICSWRFGEFMCKAYMVSTSITSFTSSIFLLIMSADRYIAVCHPISSPRYRTLHIAKVVSAIAWSTSAVLMLPVILYASTVEQEDGINYSCNIMWPDAYKKHSGTTFILYTFFLGFATPLCFILSFYYLVIRKLRSVGPKPGTKSKEKRRAHRKVTRLVLTVALIHSNPAQRDLSRLEILIFLLLGALVYSNSAVNPILYAFLSENFRKSFFKAFTCMNKQDINAQLQLEPSVFTKQGSKKRGGSKRLLTSNPQIPPLLPLNAGNNNSSTTTSSTTTAEKTGTTGTQKSCNSNGKVTAPPENLIICLSEQQEAFCTTARRGSGAVQQTDL

SEQ ID NO: 11 - DNA, 957 bp, Drosophila melanogaster
  CDS (1)..(954)
  n 956, 957: n is a or g or c or t/u, unknown, or other
atgaatctgttcgcctcgttcgctgccaataactcgttgtggctggtctggtacctgctggtcatgccgaattcggagctactgcatcagagtccgatgcgctgcgttgccctgcacataacgctacactacttcctcctgtccaattactcctggatgctctgcgagggattctatctgcacaccgtcctcgtcgccgcattcatttccgagaagaggctggtcaaatggctcatcgcattcggctggggctccccggccatcgtcatattcgtctatagcatggctcgcggtctgggcggcacgcccgaggacaatcgtcactgctggatgaaccaaaccaactaccaaaacattcttatggtgcctgtgtgtatctccatgttcctgaacctcctgttcctgtgcaacattgtgcgagtggtccttttgaaactgaatgccccggccagtattcagggcagctgcggtccatcgcgaacggttttgcaagcatttcgggcaacgctgcttttggttcctctgctcggcctccagtacatccttacgccctttcgtcctgcacccaaacatccctgggagaatacatacgaaatcatctcggcgtttacggcctcatttcaaggtctgtgcgttgccattttgttctgcttttgcaacggcgaggtgattgcccaaatgaagcgcaagtggcggatgatgtgcttcagcaaccgaccgcggaccaattcctatacagccactcaggtctcgccccatcaccaaagccaccagttgtccacggatcagcagagagctcgcagccagagcctggcgagttcgtcgtccttcctggaaggctggcgcgaccggatgccgttcctgaaacggcgacaaactatcgaccactcccgccagagccagccactcatggaggagggcggagagaccgttggccaggccaaatccgcgccagnn

SEQ ID NO: 12 - protein, 318 aa, Drosophila melanogaster
MNLFASFAANNSLWLVWYLLVMPNSELLHQSPMRCVALHITLHYFLLSNYSWMLCEGFYLHTVLVAAFISEKRLVKWLIAFGWGSPAIVIFVYSMARGLGGTPEDNRHCWMNQTNYQNILMVPVCISMFLNLLFLCNIVRVVLLKLNAPASIQGSCGPSRTVLQAFRATLLLVPLLGLQYILTPFRPAPKHPWENTYEIISAFTASFQGLCVAILFCFCNGEVIAQMKRKWRMMCFSNRPRTNSYTATQVSPHHQSHQLSTDQQRARSQSLASSSSFLEGWRDRMPFLKRRQTIDHSRQSQPLMEEGGETVGQAKSAP

SEQ ID NO: 13 - DNA, 1401 bp, Drosophila melanogaster
  CDS (1)..(1398)
atgaaggatgtgcccgacaaatatgtgacggcactctcgcacttcctcgactggcattccaatggcacggttgacatggagcgacttagtggtcccatcctaaagtccagcatcaagtcggtctattggctgtttctcatccagtacgcagctctcgccctactgggcgtggtgctcaatgtgatcatcgtggtgtacataatgtaccatcgactttacaaggatgtgacccatgcgttcatcatcaatctggccctatgccattttgtccagtgcgccctcgtcctgcccgtctcgctgatggtcatgctcatccagaactggatctttggccagtttctgtgcttctttctgcccatgctgcagtcgaagattttggagatcggcgaccctgtgcacatctatagcacgaaaaaacaacgatcaaaccttaaacagcgacagctttggaacattcccaaaataccgtcattttcccagcttcaggatatggaaatttcagcagggagcgaccacgtttcccgggaacaatgggtcatcgacggtcccaacacgcatgagaaggtgatcatcctctcagatcatccatatgccactcgagaacgacgttcatatggaaacattcgtggcgtggagaactattgcgatccggttgccacccaggacgaacgaatggaggccaatccgggcatagtcccgaaagaagaagaccctgagccttctccaaaaatcacgaatggacttatatatcaaggcaccttcaatgaagaattcgacatgctggacaatgagtcaaaagctaaagcaaatggaaacctacaagatgttaaaccagaagctccgcagcatcctttagaaaacaccgcctttcagaatacaccgcccagtattggcggacagaataatgaaaacacttcgggatacatagtcaagagggatgaaatggcaaccttgatgaatgatatagacacggatggtatcttcgattatctgaaactgcagttggacacgcagacgccaacggagcagcaagacaataaggagccccatttgctgacgtcctcaggagagaagaataaatatattggtgtggtgtccttgcatgaggcatgtccctttccaacgttcgagaagatagtcaaggatattatcgagcaggattgcaagaagataatagagaccaagaagatggtggatgagctggtcaaggtggaggtcaaaaaggagatcgaggaggtggtcaagcaggaggtcaaaaaggagatcgaggaggtggtcaagcaggaggtcaaaaaggagatcgaggaggtggtcaagaaggagatcaagaaggagatcaaggaggagatcgagaaggagatccaggcggagatcgacgatgagatcgtggaagtggactag

SEQ ID NO: 14 - protein, 466 aa, Drosophila melanogaster
MKDVPDKYVTALSHFLDWHSNGTVDMERLSGPILKSSIKSVYWLFLIQYAALALLGVVLNVIIVVYIMYHRLYKDVTHAFIINLALCHFVQCALVLPVSLMVMLIQNWIFGQFLCFFLPMLQSKILEIGDPVHIYSTKKQRSNLKQRQLWNIPKIPSFSQLQDMEISAGSDHVSREQWVIDGPNTHEKVIILSDHPYATRERRSYGNIRGVENYCDPVATQDERMEANPGIVPKEEDPEPSPKITNGLIYQGTFNEEFDMLDNESKAKANGNLQDVKPEAPQHPLENTAFQNTPPSIGGQNNENTSGYIVKRDEMATLMNDIDTDGIFDYLKLQLDTQTPTEQQDNKEPHLLTSSGEKNKYIGVVSLHEACPFPTFEKIVKDIIEQDCKKIIETKKMVDELVKVEVKKEIEEVVKQEVKKEIEEVVKQEVKKEIEEVVKKEIKKEIKEEIEKEIQAEIDDEIVEVD

SEQ ID NO: 15 - DNA, 2001 bp, Drosophila melanogaster
  CDS (1)..(1998)
atggccaatctgcgtcccctgagcgccgaacttcgtcgcattgctgaaacggagctaaacgaagtggaggagcgagtgcccgcggatctgaaagctcttcgggattggttggccaagcaacctcatctgagggcccgccaggatgaccagttcctagttggcttcttgcgaggctgcaagtttagcctggagaagaccaagtccaagctggatcacttttatactattaagacgcttatgcctgagctcttcggcaaaagattggtggacgaaaggaacctaatcttgtgcagatctggaacctatgttcgattgcccaaaccttggggaaccgatggcccccgcctgcagcttacgaactatgagaaattcgaccctaaggaattcaagttattggatctcttccgctaccaaaccatgataacggaacagtcaatccgtgaggatgaccacagcaacattagcggctacgtcgagatcgttgacatggccaaaatgagcctcagttttctggcccaactggatttcacgctcatcaaaaggatgggaatattcgctgagaaggcacagcccactcgcttgaaaggagtccatctgatcaattgccccaaggagggagtggcgttactgaatttggccaagagcctaatgcccagcaagctccagcagcggtttcacgtgtataaaaatctcgagcaattaaacgaggtgataccacgcgaatacctgccggaagaatatggtggcaacaatggacgtatagccgacattcaggcggaagcggagaagaaattgctgtcctatgagagctattttgctgaggattcccagtacggatttgacttcggcaaatgggacttcccagccgagcgcatctggctgcacaagcccaatggggagatcacctggaagatctgcacttttctaccactgattgcttttggtctgtatggaaacttcagcatggtttatgtgatagccaccaaccgttctttgcgctcgcccaccaacttgattattgcaaatatggccgttgcggatctactgaccctggccatttgtcctgccatgttcatggtgaacgacttctatcagaactaccagttgggatgcgtgggctgcaagctggagggctttttggtggtggtcttcctcatcacagccgtgctcaatctctcggtagtcagctacgatcgtctcacggccatcgtgctgccaatggagacgcgcctcaccataaggggcgtccagatagtggtggtctgcacctgggtctcgggtatcctattggcatcaccattggctttctatcgctcttacagggtgcgagtgtggaagaactttacggagcgatattgcaaggagaacacctctgtgctgcccaagtactggtatgttctgatcaccattctggtgtggctgccattaggcatcatgcttatctgctacatagccatattttataagcttgatcggtatgaaaagcgggtcctaagtcgcgagaacccgctgacagtgagctataaacgcagcgtggccaagacactgttcattgtggtcgttgtctttgcagctctacgactgccctttacaatcctggttgtactacgggaaaaatacttcggtgaagatgtctctgtcagcagtggaatgcagctattctggtacatatcacagtatttgatgtttctgaacgccgccgtcaatccgctgatctatggcttcaacaatgagaactttagaagggcctactaccaaatttcttgggttcgccgatggcgagacgccacccaaatgaagaagttttctcgatcaccagaccactgctgctactgtgcctttatgaagaatggaaaacgaacctcagaggcagcacaaaaggcaggaaatttagaaaaagacataagcaaggatatgtcgtcggcacaacaatcggccaagtcaacgaagatagttgaaaatgaatttgtatcagaaatcgaggcagacggatttatataa

SEQ ID NO: 16 - protein, 666 aa, Drosophila melanogaster
MANLRPLSAELRRIAETELNEVEERVPADLKALRDWLAKQPHLRARQDDQFLVGFLRGCKFSLEKTKSKLDHFYTIKTLMPELFGKRLVDERNLILCRSGTYVRLPKPWGTDGPRLQLTNYEKFDPKEFKLLDLFRYQTMITEQSIREDDHSNISGYVEIVDMAKMSLSFLAQLDFTLIKRMGIFAEKAQPTRLKGVHLINCPKEGVALLNLAKSLMPSKLQQRFHVYKNLEQLNEVIPREYLPEEYGGNNGRIADIQAEAEKKLLSYESYFAEDSQYGFDFGKWDFPAERIWLHKPNGEITWKICTFLPLIAFGLYGNFSMVYVIATNRSLRSPTNLIIANMAVADLLTLAICPAMFMVNDFYQNYQLGCVGCKLEGFLVVVFLITAVLNLSVVSYDRLTAIVLPMETRLTIRGVQIVVVCTWVSGILLASPLAFYRSYRVRVWKNFTERYCKENTSVLPKYWYVLITILVWLPLGIMLICYIAIFYKLDRYEKRVLSRENPLTVSYKRSVAKTLFIVVVVFAALRLPFTILVVLREKYFGEDVSVSSGMQLFWYISQYLMFLNAAVNPLIYGFNNENFRRAYYQISWVRRWRDATQMKKFSRSPDHCCYCAFMKNGKRTSEAAQKAGNLEKDISKDMSSAQQSAKSTKIVENEFVSEIEADGFI

SEQ ID NO: 17 - DNA, 1233 bp, Drosophila melanogaster
  CDS (1)..(1230)
atggaaacggacttggccatgaacatcggcctggacacttccggtgaagcccccacagccctacccccgatgccgaatgtcaccgagacgctgtgggatctggccatggtggtcagtcagtccacacagtggcccctcctggacaccggctcgtccgagaacttcagcgaactggtcaccaccgagacaccctacgtgccctacggccgtcgaccggaaacctacatcgtgcccatcctctttgccctgatcttcgtggtcggtgtcctgggcaatggcactttgattgtggtcttcctcagtgtgcgacagatgcgaaatgttccaaacaccggcggccagagaacgtggcaaacaaagtgccacatgcagcttatttatggtaagcggcgtggttggataaagtcggaattactgcatgagatcacggaccgaattggatgtgccactactgagcaccattcgttcgctggcctcaagttttacacagttgaatattggccatatggcagcttcctgtgcagcctgtcggagttcatgaaggatgtgtccatcggagtgtcagtgtttacgctgactgcattatccggcgatcggtactttgccatcgtggatcctttgaggaagttccacgctcatggtggtggtcgtcgggccaccagaatgaccttggccaccgcggtttccatttggctgctggccatactctgcggacttccagcgctaattggaagtaacctgaagcatctcggcataaacgaaaagtccatagtcatttgctatccgtatccggaggagtggggcatcaactacgccaagtccatggtcctgttgcacttcctggtctactacgccattcctttggtcgtcatagcagttttctacgtgctcatcgccctccatttgatgtattcggccagtgttcctggtgaaattcagggagctgtgcgtcaggttcgtgcccgtcgcaaagtggctgttactgtgctggccttcgttgtcatctttggaatttgctttctgccctaccacgtcttcttcctctggttctacttctggcccaccgcccaggatgattacaatgccttctggcatgtgctgcgcatcgtcgcctactgcatgtcctttgccaacagctgtgcgaatcctgtggccctatacttcgtgagcggcgcctttcgcaagcatttcaataggtatttgttcgtttacatcaatccaccagccgcataa

SEQ ID NO: 18 - protein, 410 aa, Drosophila melanogaster
METDLAMNIGLDTSGEAPTALPPMPNVTETLWDLAMVVSQSTQWPLLDTGSSENFSELVTTETPYVPYGRRPETYIVPILFALIFVVGVLGNGTLIVVFLSVRQMRNVPNTGGQRTWQTKCHMQLIYGKRRGWIKSELLHEITDRIGCATTEHHSFAGLKFYTVEYWPYGSFLCSLSEFMKDVSIGVSVFTLTALSGDRYFAIVDPLRKFHAHGGGRRATRMTLATAVSIWLLAILCGLPALIGSNLKHLGINEKSIVICYPYPEEWGINYAKSMVLLHFLVYYAIPLVVIAVFYVLIALHLMYSASVPGEIQGAVRQVRARRKVAVTVLAFVVIFGICFLPYHVFFLWFYFWPTAQDDYNAFWHVLRIVAYCMSFANSCANPVALYFVSGAFRKHFNRYLFVYINPPAA

SEQ ID NO: 19 - DNA, 2586 bp, Drosophila melanogaster
  CDS (1)..(2583)
atggagctggacgataatttaataagcagcctgcccgagggactcagcaaattgtcgcaactgcaggagctttccatcaccagcaatcgactgcgctggataaacgacacggaactgcccaggagcatgcaaatgctggacatgagggccaatccactgtccacaatttcgccaggggcgtttcggggcatgtccaagttgcggaagctgattttatcggatgtgcgaactttgcgctcgtttcccgaacttgaggcctgccatgcgctggaaatactgaaactggatcgtgccggcattcaggaggtgccagcgaatctatgccgccaaacgccaagactgaagagtttagatctatcgagcaatcaaattgaaaagattcagggaaagcccttcaatggattgaagcaattgaacgatctgttgctctcctacaatcgaatcaaggcactgccacaagatgcctttcagggcattccgaagctccaacttctcaagcagttgggtgcttccatgcatgatccttgggagacggctattaacttcaacgaagagcagctgcaaacacaaacgggaggccaaatcgccaccagctacatggaggagtacttcgaggagcacgatgtgagtggtcctgccacgggatacggctttggtactggactattctctggtatgtccacagaggactttcaaccgggttcggtgcagtgcctgccaatgccaggaccttttctaccctgcgccgatctcttcgattggtggactctgcgctgcggagtgtgggtggtttttctgttgtctctcctgggcaacggaacggtggtgtttgtgctgctctgctcgcgatcaaagatggatgtgccccgatttctagtctgtaatctggccgcggccgacttcttcatgggcatctacctgggtattctggccattgtggatgcggcgacgttgggcgaatttcgcatgtttgccattccttggcagatgtccgtgctctgccagttgtccggttttctggccgtactcagctcagaattatccgtttacacgttggctgtgatcactttggagcgcaattacgccatcactcatgccattcacctgaacaagaggctctccttgaagcaggcgggatatataatgagtgtaggatgggtttttgccctgatcatggctttgatgcctttggttggtgtttcggactacaggaagtttgccgtgtgtttgccattcgagaccaccactggaccggccagtttgacctatgtaatctcactgatgttcatcaacggatgcgcatttctcactctgatgggttgctacctgaagatgtactgggccataaggggcagccaggcatggaacacgaatgattcgcgaattgccaagcgaatggctctgttggtctttactgacttcctctgctggtcacctatcgccttcttctcgatcactgccatctttggcctgcaactgatttcgctggagcaggccaagatctttacggtatttgtgctgcccttgaatagttgctgcaatccctttctctacgccataatgaccaagcagttcaagaaggactgtgtgacgttgtgcaaacatttcgaagaatcccgcgtcgtgggaggcggtggtccgggtggacgtggtgcagtggcaagaaccaagaggggtgatctcccgccaccactcttaccagctgcagcggtggcacatccgccaggctgtcgatgtctacggatgctgcccagcgaaatgcccaactggcataaaatggagcagacaccgagcatgtggcagagattgagaacattctgctgcggggagaaccgaaggcgtcgcaaacagcgacggcaaccgcaacagcgacgccagagagcctatactgccgcagccgccaatccgtatcagtatcagtttgctgaactgcgtcagcaacgccagaatcgagccagttccatttccagcgagaatttctgcagctcaagatcatccagctggcgacatggtccaccatcctcagcacccgtgccaccgggtaactgcagcatgccgctgaagatgctggagccacatgctcatccgcacggtcatgggcgcagacgacactccgcctggctgatcacgcggaaaacgtcgcaggactcgaacttgtccagctcgcgcaatgactcctccgcctcggccaccaccgccagcacctccacgttccggttatcccggtcaagtgccggcagcagcacacccctgccctcgatcatagcccacaacgggaaggcccagttggatgcagttaaaccacgattggtccgccaggaagcagtccaggaggaagaggactcatcaccacctcgcctgggagtacgcttcctacccaccataccctcggctgccgacagttctgtcgtcatggaagatggcgactcggccaacacgggagtagccagttttctaggcatgccacttcctggggcgtccagtggcttccttatagctccaaccaccgctgccaccacctccccacccccggtggtcctgcagccggcaaagcctcctcccgatcccaacgatgcaccactatga

SEQ ID NO: 20 - protein, 861 aa, Drosophila melanogaster
MELDDNLISSLPEGLSKLSQLQELSITSNRLRWINDTELPRSMQMLDMRANPLSTISPGAFRGMSKLRKLILSDVRTLRSFPELEACHALEILKLDRAGIQEVPANLCRQTPRLKSLDLSSNQIEKIQGKPFNGLKQLNDLLLSYNRIKALPQDAFQGIPKLQLLKQLGASMHDPWETAINFNEEQLQTQTGGQIATSYMEEYFEEHDVSGPATGYGFGTGLFSGMSTEDFQPGSVQCLPMPGPFLPCADLFDWWTLRCGVWVVFLLSLLGNGTVVFVLLCSRSKMDVPRFLVCNLAAADFFMGIYLGILAIVDAATLGEFRMFAIPWQMSVLCQLSGFLAVLSSELSVYTLAVITLERNYAITHAIHLNKRLSLKQAGYIMSVGWVFALIMALMPLVGVSDYRKFAVCLPFETTTGPASLTYVISLMFINGCAFLTLMGCYLKMYWAIRGSQAWNTNDSRIAKRMALLVFTDFLCWSPIAFFSITAIFGLQLISLEQAKIFTVFVLPLNSCCNPFLYAIMTKQFKKDCVTLCKHFEESRVVGGGGPGGRGAVARTKRGDLPPPLLPAAAVAHPPGCRCLRMLPSEMPNWHKMEQTPSMWQRLRTFCCGENRRRRKQRRQPQQRRQRAYTAAAANPYQYQFAELRQQRQNRASSISSENFCSSRSSSWRHGPPSSAPVPPGNCSMPLKMLEPHAHPHGHGRRRHSAWLITRKTSQDSNLSSSRNDSSASATTASTSTFRLSRSSAGSSTPLPSIIAHNGKAQLDAVKPRLVRQEAVQEEEDSSPPRLGVRFLPTIPSAADSSVVMEDGDSANTGVASFLGMPLPGASSGFLIAPTTAATTSPPPVVLQPAKPPPDPNDAPL

SEQ ID NO: 21 - DNA, 606 bp, Drosophila melanogaster
  CDS (1)..(603)
atgttcaactacgaggagggggatgccgaccaggcggccatggctgcagcggctgcctatagggcactgctcgactactatgccaatgcgccaagtgcggcgggtcacatagtgtcgctcaacgtggcaccctacaatggaactggaaacggaggcactgtctccttggcgggcaatgcgacaagcagctatggcgatgatgatagggatggctatatggacaccgagcccagtgacctggtcaccgaactggccttctccctgggcaccagttcaagtccaagtcccagttccacacccgcttccagctccagtacttccactggcatgcccgtctggctgatacccagctatagcatgattctgctgttcgccgtgctgggcaacctgctggtcatctcgacgctggtgcagaatcgccggatgcgtaccataaccaacgtgttcctgctcaacctggccatatcggacatgctgctgggcgtgctctgcatgcccgtcaccctggtgggcaccctgctgcgaaacttcatctttggcgagttcctctgcaagctctttcagttctcgcaaggttcttgggctcttgggttcctgcgcatttag

SEQ ID NO: 22 - protein, 201 aa, Drosophila melanogaster
MFNYEEGDADQAAMAAAAAYRALLDYYANAPSAAGHIVSLNVAPYNGTGNGGTVSLAGNATSSYGDDDRDGYMDTEPSDLVTELAFSLGTSSSPSPSSTPASSSSTSTGMPVWLIPSYSMILLFAVLGNLLVISTLVQNRRMRTITNVFLLNLAISDMLLGVLCMPVTLVGTLLRNFIFGEFLCKLFQFSQGSWALGFLRI

SEQ ID NO: 23 - DNA, 1224 bp, Drosophila melanogaster
  CDS (1)..(1221)
atggcctccgtggccgtttcgtcctggaccttggtggccatatcctgtgagcgctactacgcgatatgccatccactgcgctcgcgatcctggcagacaatcagtcacgcctacaagatcatcggcttcatctggctgggcggcatcctctgcatgacgcccatagcggtctttagtcaattgatacccaccagtcgaccgggctactgcaagtgccgtgagttttggcccgaccagggatacgagctcttctacaacatcctgctggacttcctgctgctcgtcctgccgcttctcgtcctctgcgtggcctacatcctcatcacgcgtaccctgtacgtaggcatggccaaggacagcggacgcatcctgcagcaatcgctgcctgtttccgctacaacggccggcggaagcgcaccgaatccgggcaccagcagcagtagtaactgcatcctggtcctgaccgccaccgcagtctataatggttggcggaggatcaaccaatatggcaacgaccaccttgacaacgagaccaacggctccaactgtgatcaccaccaccacgacgaccacggtgacgctggccaagacctcctcgcccagcattcgcgtccacgatgcggcacttcgcaggtaggattcgtgtccgataaccggtctgccaatcctattccctattcccatttctccacaatccacaatccactatccaggtccaacgaggccaagaccctggagagcaagaagcgtgtggtcaagatgctgttcgtcctggtgctggagtttttcatctgctggactccgctgtacgtgatcaacacgatggtcatgctgatcggaccggtggtgtacgagtatgtcgactacacggccatcagtttcctccagctgctggcctactcatccagctgctgcaatccgatcacctactgcttcatgaacgccagcttccggcgcgcctttgtcgacaccttcaagggtctgccctggcgtcgtggagcaggtgccagcggaggcgtcggtggtgctgctggtggaggactctccgccagccaggcgggcgcaggcccgggcgcctatgcgagtgccaacaccaacattagtctcaatcccggcctagccatgggtatgggcacctggcggagtcgctcacgccacgagtttctcaatgcggtggtgaccaccaatagtgccgccgccgccgtcaacagtcctcagctctaa

SEQ ID NO: 24 - protein, 407 aa, Drosophila melanogaster
MASVAVSSWTLVAISCERYYAICHPLRSRSWQTISHAYKIIGFIWLGGILCMTPIAVFSQLIPTSRPGYCKCREFWPDQGYELFYNILLDFLLLVLPLLVLCVAYILITRTLYVGMAKDSGRILQQSLPVSATTAGGSAPNPGTSSSSNCILVLTATAVYNGWRRINQYGNDHLDNETNGSNCDHHHHDDHGDAGQDLLAQHSRPRCGTSQVGFVSDNRSANPIPYSHFSTIHNPLSRSNEAKTLESKKRVVKMLFVLVLEFFICWTPLYVINTMVMLIGPVVYEYVDYTAISFLQLLAYSSSCCNPITYCFMNASFRRAFVDTFKGLPWRRGAGASGGVGGAAGGGLSASQAGAGPGAYASANTNISLNPGLAMGMGTWRSRSRHEFLNAVVTTNSAAAAVNSPQL

SEQ ID NO: 25 - DNA, 1258 bp, Drosophila melanogaster
  CDS (1)..(1257)
atgtccgctggcaatatgagccatgatcttggaccgccacgcgatccgctggccatcgtgatacccgtaacggtggtctactccctcattttcataaccggtgtagtgggcaacataagtacctgcattgtgattaagaagaaccgttcaatgcacacggccacgaattactacctcttttcgctggccatctcggatttcctgctcctgttgtcgggcgttccgcaggaggtgtcctacatctggtccaagtacccgtacgtgtttggggagtacatctgcatcggacgtggtctgttggcggagacatcggcgaatgccacggtgctaacgattacggccttcacggtggagcggtatattgccatttgccatccgtttctgggccaggccatgagtaaactcagtcgcgccattcgcatcatcgtcctggtttggattatggccatagttacggccattccgcaggctgcccaatttggaatcgagcactattcgggagtggagcaatgcggcatagtgcgggtcatagtgaagcactcattccagctgtccacgttcatattcttcctggctccgatgtctattatcctagtgttgtacctacttatcggtgtgcacctgtatcgatccactttggtggagggtcctgcctcggttgccagacggcagcagctgaagagtgtgcccagtgatacgatcctatatcgctatggtggatccggtaccgctatgagtttcaacggcggaggaagtggagcagggacagcgggcttgatgggcggctcgggggctcaacttagctcggtgagaggtcggctcaatcactatggcacccggcgagtactcaggatgctagtggccgtggtggtgtgcttcttcctctgctgggcccccttccacgcccagcgactgattgccatctacgcccctgcacggggggccaaactgcgggatcagcacgagtttgtctacacggtgatgacctatgtctccggtgtcctctactacttgtccacctgcatcaacccgctgttatataacattatgagccacaagttccgagaggcattcaaggccgttctgtttggcaagaaggtatcgaagggttcgctgaattcgcgaaacaacatcgaatcgcgccgcctgaggagggcactaaccaattccagtcaaacgcagcgcttctccattgagtcggcggagcagccgaaaccgtcgataatgcaggcaagtgccccctataatcaatggatag

SEQ ID NO: 26 - protein, 419 aa, Drosophila melanogaster
MSAGNMSHDLGPPRDPLAIVIPVTVVYSLIFITGVVGNISTCIVIKKNRSMHTATNYYLFSLAISDFLLLLSGVPQEVSYIWSKYPYVFGEYICIGRGLLAETSANATVLTITAFTVERYIAICHPFLGQAMSKLSRAIRIIVLVWIMAIVTAIPQAAQFGIEHYSGVEQCGIVRVIVKHSFQLSTFIFFLAPMSIILVLYLLIGVHLYRSTLVEGPASVARRQQLKSVPSDTILYRYGGSGTAMSFNGGGSGAGTAGLMGGSGAQLSSVRGRLNHYGTRRVLRMLVAVVVCFFLCWAPFHAQRLIAIYAPARGAKLRDQHEFVYTVMTYVSGVLYYLSTCINPLLYNIMSHKFREAFKAVLFGKKVSKGSLNSRNNIESRRLRRALTNSSQTQRFSIESAEQPKPSIMQASAPYNQWI

SEQ ID NO: 27 - DNA, 1602 bp, Drosophila melanogaster
  CDS (1)..(1599)
atggaaggtggatggtggcgaggaggaggaggaggaggacgtttgggcgggaaagccatcatggaaggacactcgacaccaaatggggcagctgcaagccacagaaacaacagcacaaggaccaatatcgcgaccaacggctgtgcccattcgggcatcctgttatttgtgctgacagctatgacactaacgagcttaataacgcccacagagcagctggcagtggcgccaaatggcaccacactgcatcaactggagtccgtggagtccgagtcgtatccatccataaatggcactcagaatgaaacaatggtgaccagtgtgcgaccccacctggaccacaggaatcgaccgacgcagcagaatggcagtcactatttggagtacgacgatgatggcccggactgttcgtacagctacaacttcatcctgaagctcatcacgatgatcctgtacgcactggtctgcatcattggactctttggcaacaccctggtgatctatgtggtgatgaggttctccaagatgcagaccgtaaccaacatatacatactgaatctggctatcgcggatgagtgcttcctgatcggcattcccttcctgctctacacaatgcaggtgggcaactggcccttcggcaactatatgtgcaaggcctacatggtgagcacctcgatcacctccttcacctcctcgatcttcctgttgatcatgtcggcggatcgctacatagccgtttgccatcccatatcctcgcctcgctaccgaacgccctttgtatccaagttggtttcggccttcgcctggatgacatccgtgctgctgatgctgccggttatcctttttgccagcaccgtgcagtcgagcaacggcaatgtgtcctgcaacatcgagtggccagacactcagaactcgcacaccgactccaccttcattttgtactcgctggtcttgggattcgccactccactgacttttatcctggtgttctactgcctggtgatcaggaaacttcacaccgtgggaccgaagcacaagtctaaggagaagaagcgctctcacaggaaggtcaccaagttggtgctcacggtggctttgatcagctccgctcctcaacgctgtgcatctcgtctggagctggccgtcttcctggcctgcggatgcctcagctactccaactcggccatgaacccaatactgtacgcctttttgagcgataacttcaagaagagcttcatgaaggcctgcacgtgtgctgcccgcaaggatgtgaatgcccagttgcagctggagaacagtttcttccccaagtttggcaagggcaggcaatcggagcgtcttcttggtggcaatggaaaaggtggcgcccagcgtggggcattaaccaagaagaagtgcttggcgacgagaaacaacaatgctccgatggccactacaacgacgacgacaaccaccacaacgggcacggatgcagtgacctgtctccagccgccagtacaccaggtgccagccgagatccaggtgggaaatccggccaccgtgctggtggtcaatgctgagaccaacaactgtaaaccgcccgtgctccacacggacttataa

SEQ ID NO: 28 - protein, 533 aa, Drosophila melanogaster
MEGGWWRGGGGGGRLGGKAIMEGHSTPNGAAASHRNNSTRTNIATNGCAHSGILLFVLTAMTLTSLITPTEQLAVAPNGTTLHQLESVESESYPSINGTQNETMVTSVRPHLDHRNRPTQQNGSHYLEYDDDGPDCSYSYNFILKLITMILYALVCIIGLFGNTLVIYVVMRFSKMQTVTNIYILNLAIADECFLIGIPFLLYTMQVGNWPFGNYMCKAYMVSTSITSFTSSIFLLIMSADRYIAVCHPISSPRYRTPFVSKLVSAFAWMTSVLLMLPVILFASTVQSSNGNVSCNIEWPDTQNSHTDSTFILYSLVLGFATPLTFILVFYCLVIRKLHTVGPKHKSKEKKRSHRKVTKLVLTVALISSAPQRCASRLELAVFLACGCLSYSNSAMNPILYAFLSDNFKKSFMKACTCAARKDVNAQLQLENSFFPKFGKGRQSERLLGGNGKGGAQRGALTKKKCLATRNNNAPMATTTTTTTTTTGTDAVTCLQPPVHQVPAEIQVGNPATVLVVNAETNNCKPPVLHTDL

SEQ ID NO: 29 - DNA, 1347 bp, Drosophila melanogaster
  CDS (1)..(1344)
atgtttctatataagaattgttacaattttagatttgagtgctatgaattgaatttcgaatgcctggagtataagttctataaattaaataccaagccacgcattttgagctgtgaaactgccttaaaagtcgaaagaaatatttttcgaagtgcaggtccttcatcttggcagcaatttcctgctggaataacggtggctgtctgtcttccatcacgtctgggtgtgtggaccgaacagtttgctgtgctctggatcctgttcaccgtcatcgttctgggcaattcagctgttctgttcgtgatgttcatcaacaagaatcgcaagtcgcggatgaactacttcattaaacagctggcattggcagatctgtgcgtgggactgctcaacgtcctcaccgacatcatatggcgcatcacgatttcgtggcgggcaggcaacctggcctgcaaggccatccgcttctcgcaggtctgcgtcacatactcgtccacctacgtgctggtggccatgagcatcgacagatacgatgccatcacacaccccatgaacttctcaaagtcgtggaaaagagcccgtcacctggtggctggcgcatggctcatctcggcgttgttttcgcttcccatcctggttttgtacgaggagaagctcatccaaggacatccgcaatgctggattgagttgggttcaccgatcgcctggcaggtgtacatgagcctggtgtcggccactctatttgccattcctgcgctgatcatatctgcctgctatgcgatcatcgtaaagacgatttgggcaaagggttccatttttgtacccacggaacgtgctggttttggagctgcacctgccaggagggccagctcgaggggcattattccacgggcaaaggtcaaaacggtcaagatgacattgaccatcgtgtttgtgttcatcatctgctggtcgccgtatatcatcttcgatctgctgcaggtctttggccagattccacactcacagaccaacattgccatcgccaccttcatccaaagtctggcaccgctgaactcggcggcgaatccactaatctattgcctcttctcatcgcaggtctttcgcacattaagtcgctttccgccttttaagtggttcacatgctgctgcaagtcataccgcaacaactcgcagcaaaaccgctgccacacggttggtcgtcggcttcacaacagttgcgattcgatgaggacactgaccacttcgttgacggtttcccgaaggtccaccaacaagacgaacgcccgtgtggtaatctgcgaacgtcccaccaaggtggttaccgtgccagccatgtcggaggtatga

SEQ ID NO: 30 - protein, 448 aa, Drosophila melanogaster
MFLYKNCYNFRFECYELNFECLEYKFYKLNTKPRILSCETALKVERNIFRSAGPSSWQQFPAGITVAVCLPSRLGVWTEQFAVLWILFTVIVLGNSAVLFVMFINKNRKSRMNYFIKQLALADLCVGLLNVLTDIIWRITISWRAGNLACKAIRFSQVCVTYSSTYVLVAMSIDRYDAITHPMNFSKSWKRARHLVAGAWLISALFSLPILVLYEEKLIQGHPQCWIELGSPIAWQVYMSLVSATLFAIPALIISACYAIIVKTIWAKGSIFVPTERAGFGAAPARRASSRGIIPRAKVKTVKMTLTIVFVFIICWSPYIIFDLLQVFGQIPHSQTNIAIATFIQSLAPLNSAANPLIYCLFSSQVFRTLSRFPPFKWFTCCCKSYRNNSQQNRCHTVGRRLHNSCDSMRTLTTSLTVSRRSTNKTNARVVICERPTKVVTVPAMSEV

SEQ ID NO: 31 - DNA, 1449 bp, Drosophila melanogaster
  CDS (1)..(1446)
atgaacgagggcgttattgacatgcgaaacgtggacgaggagctggcggagaaggaggagctgatggccaccgtcgtcagtgccacgatggccacaaatcaggaggagaaccgactcttttgccccctgaactttgacggatatctctgctggccaaggactccggccggcactgtgctgagtcaatattgtcccgatttcgtcgaaggattcaacagaaaatttctggcccacaagacctgcctggagaacggctcctggtaccgtcatccggttagcaatcagacctggtccaattacaccaactgcgtggactatgaggacctggagtttcgccagttcatcaacgagctgtacgtgaagggttatgccctctccctgctggccctgctcatatcgattattattttcttgggcttcaaatccctgcgctgcacccgcatccgcatccatgtacatctgttcgcctcgctggcctgcacctgtgtggcctggatattatggtatcgtctggtggtggagcgttccgaaaccatagccgaaaatccggtaagatgcactgagaaaagtaactatgaaaataacatagccctacaagtagctaggcaattatacaaaaccataaacataactaactttcagctgtggtgcattgggctgcatctggtggtgcactactttatgttggtcaactatttctggatgttttgcgagggcctgcacctgcacctggtgctcgttgtggtgagttatccttactcgtctggcaatctggcaattgcctgcacaagtctgcattctttatgggcgacttctgtgcatggcagatgtgatttcctgcagccgctgacatggcaatggttcacccccagcaatttgtctgagagcaaatcgcaacgttggccaactgctttacgtctgcgtcaagttgagatgtcaggaatgtccgttcagatcctgacgccccttcttctttccattcgccaggtctttgtcaaggacacgattgtgatgcgctggtttattgtcatcagctggttttcacccatacccatagccatcgtctacgtttttctgatcaacgtactgcgagtgatcgtgcgaaagttacatccccagtccgcccagccggcaccgttggccatccggaaggcggttcgggccaccattattctggtaccgctgttcggactgcaacacttcctgctgccctaccgtccggatgccggcacccagctggaccacttctatcagatgctatcggtggtgttggtcagcctgcagggctttgtggtctccttcctgttctgcttcgccaatcacgatgtcacctttgccattcgcacgctgctgaacaagttgctgcccagtttagtgaccccgccgccggccgggagtaatactggacagatggccacgaccacgcccagccgagagttgggcgtttaa

SEQ ID NO: 32 - protein, 482 aa, Drosophila melanogaster
MNEGVIDMRNVDEELAEKEELMATVVSATMATNQEENRLFCPLNFDGYLCWPRTPAGTVLSQYCPDFVEGFNRKFLAHKTCLENGSWYRHPVSNQTWSNYTNCVDYEDLEFRQFINELYVKGYALSLLALLISIIIFLGFKSLRCTRIRIHVHLFASLACTCVAWILWYRLVVERSETIAENPVRCTEKSNYENNIALQVARQLYKTINITNFQLWCIGLHLVVHYFMLVNYFWMFCEGLHLHLVLVVVSYPYSSGNLAIACTSLHSLWATSVHGRCDFLQPLTWQWFTPSNLSESKSQRWPTALRLRQVEMSGMSVQILTPLLLSIRQVFVKDTIVMRWFIVISWFSPIPIAIVYVFLINVLRVIVRKLHPQSAQPAPLAIRKAVRATIILVPLFGLQHFLLPYRPDAGTQLDHFYQMLSVVLVSLQGFVVSFLFCFANHDVTFAIRTLLNKLLPSLVTPPPAGSNTGQMATTTPSRELGV

SEQ ID NO: 33 - DNA, 1521 bp, Drosophila melanogaster
  CDS (1)..(1518)
  n 1521: n is a or g or c or t/u, unknown, or other
atgtacgcctccttgatggacgttggccagacgttggcagccaggctggcggatagcgacggcaacggggccaatgacagcggactcctggcaaccggacaaggtctggagcaggagcaggagggtctggcactggatatgggccacaatgccagcgccgacggcggaatagtaccgtatgtgcccgtgctggaccgcccggagacgtacattgtcaccgtgctgtacacgctcatcttcattgtgggagttttgggcaacggcacgctggtcatcatcttctttcgccaccgctccatgcgcaacatacccaacacatacattctttcactggccctggctgatctgttggttatattggtgtgtgtacctgtggccacgattgtctacacgcaggaaagctggccctttgagcggaacatgtgccgcatcagcgagttctttaaggacatatccatcggggtgtccgtgtttacactgaccgccctttccggcgagcggtactgcgccattgtaaatcccctacgcaagcttcagaccaagccgctcactgtctttactgcggtgatgatctggatcctggccatcctactgggcatgccttcggttcttttctccgacatcaagtcctaccctgtgttcacagccaccggtaacatgaccattgaagtgtgctccccatttcgcgacccggagtatgcaaagttcatggtggcgggcaaggcactggtgtactacctgttgccgctgtccatcattggggcgctatacatcatgatggccaagcggctccatatgagcgcccgcaacatgcccggcgaacagcagagcatgcagagccgcacccaggctagggcccgactccatgtggcgcgcatggtggtagcattcgtggtggtgttcttcatctgcttcttcccgtaccacgtgtttgagctgtggtaccacttctacccaacggctgaggaggacttcgatgagttctggaacgtgctgcgcatcgttggattttgcacgagcgcagtaaaactcaaaaataatgtgtcagcaatttggtggcttaattcactgccgctcgtcgtcaaagccatttgcattctggccaaatggcttcctaaactcgtgcgtcaaccccgtggcctctactgcgtgtccggggtgtttcggcagcactttaatcgctacctctgctgcatctgcgtcaagcggcagccgcacctgcggcagcactcaacggccactggaatgatggacaataccagtgtgatgtccatgcgccgctccacgtacgtgggtggaaccgctggcaatctgcgggcctcgctgcaccggaacagcaatcacggagttggtggagctggaggtggagtaggaggaggagtagggtcaggtcgtgtgggcagctttcatcggcaggactcgatgcccctgcagcacggaaatgcccacggaggtggtgcgggcgggggatcctccggacttggagccggcgggcggacggcggcagtgagcgaaaagagn

SEQ ID NO: 34 - protein, 506 aa, Drosophila melanogaster
MYASLMDVGQTLAARLADSDGNGANDSGLLATGQGLEQEQEGLALDMGHNASADGGIVPYVPVLDRPETYIVTVLYTLIFIVGVLGNGTLVIIFFRHRSMRNIPNTYILSLALADLLVILVCVPVATIVYTQESWPFERNMCRISEFFKDISIGVSVFTLTALSGERYCAIVNPLRKLQTKPLTVFTAVMIWILAILLGMPSVLFSDIKSYPVFTATGNMTIEVCSPFRDPEYAKFMVAGKALVYYLLPLSIIGALYIMMAKRLHMSARNMPGEQQSMQSRTQARARLHVARMVVAFVVVFFICFFPYHVFELWYHFYPTAEEDFDEFWNVLRIVGFCTSAVKLKNNVSAIWWLNSLPLVVKAICILAKWLPKLVRQPRGLYCVSGVFRQHFNRYLCCICVKRQPHLRQHSTATGMMDNTSVMSMRRSTYVGGTAGNLRASLHRNSNHGVGGAGGGVGGGVGSGRVGSFHRQDSMPLQHGNAHGGGAGGGSSGLGAGGRTAAVSEK

SEQ ID NO: 35 - DNA, 1578 bp, Drosophila melanogaster
  CDS (1)..(1575)
atgacgtcctttattggcggcccaaggagtcgtcgccacacgcacgaaatgattatgacgatgatgcagacggtaagggcatggcaacaggaatcggatgtggagcaccgaaaacaacataagcagcgatggcgacctgatggggcacacatatcagcggcttacgacctcaacagtgataatgacgacggccaccacagggtggtgcataatcaaaacaacggcagcccaaacagctctccgaatcagagcacttccgccttccgacagcggcaaccgcaccacccacccaccggacaacaaccgccccggctgccctgcaccgtaacccacttctccgcacactggaaaacgctgctcatcctgctcaccctgctctccgcttccacgctgacagcctccgccaatgtcaccagcaccatcagtccgcccatcaacggcagcagcaccgattacatcctgctctatggcgaatccaccacatcccttgtgcccgccctgaccaccggactctcgggggacggaagtggtgcggttatcgaggacgaggaggatgccgagaaggccagcgagtacatctttgatcgcaccgatgtgcgcataatattcatcacactctacacactggtgttttgctgctgtttctttggtaacctccttgttattttggtggtcacattatcacgacgactgcgctcaataaccaactttttcctcgccaacttggcgtttgccgacttctgtgttggactcttttgtgtgatgcagaatctgtccatctatctgatagaaagctgggtctttggcgagttcctctgccgcatgtaccagtttgtccactccctgagctacacggcctccattttcatcctggttgtcatctgtatggagcgctactttgccatcgtacaccccattacctgcaaacagatcctaactgcggctcgtctgaggatggtcattgtcacagtttggattacatcggcagtttactccacgcccaagtttgttttcagcaagaccattaagaacatccacacgcaggacgggcaggaggaggagatatgtgtcctggaccgtgagatgttcaactccaagttgctggacatgattaactttgtgcttctctacgttatgccgctcttggtgatgacggtgctgtacagcaaaatcgccatcgccttgtggcgcagttcgcgcggcctcacgccgcatgtggtgcagcatcagcatcagcagccgcagcagccatcctgccaggacattggcatgggtatgcacaacagcatgtaccatcaccatccgcatcatcatcaccaccaccaccagcatcaccagctgcaatcggcggcttcgtcggcaggagtggtgggagtgggtttgggtggtggtggtgggggcggtcctggtccgtcactcgcttccggcggcagcagcaccacgtccttgtcccgcaaacagagcagtaaatatgagaagcgcggcgtcagcatcacggagagccaggtgagctgccaggagaacactgcatcagaaaaatcgccggttgagcacggaggagtagccttaaaagcctaa

SEQ ID NO: 36 - protein, 525 aa, Drosophila melanogaster
MTSFIGGPRSRRHTHEMIMTMMQTVRAWQQESDVEHRKQHKQRWRPDGAHISAAYDLNSDNDDGHHRVVHNQNNGSPNSSPNQSTSAFRQRQPHHPPTGQQPPRLPCTVTHFSAHWKTLLILLTLLSASTLTASANVTSTISPPINGSSTDYILLYGESTTSLVPALTTGLSGDGSGAVIEDEEDAEKASEYIFDRTDVRIIFITLYTLVFCCCFFGNLLVILVVTLSRRLRSITNFFLANLAFADFCVGLFCVMQNLSIYLIESWVFGEFLCRMYQFVHSLSYTASIFILVVICMERYFAIVHPITCKQILTAARLRMVIVTVWITSAVYSTPKFVFSKTIKNIHTQDGQEEEICVLDREMFNSKLLDMINFVLLYVMPLLVMTVLYSKIAIALWRSSRGLTPHVVQHQHQQPQQPSCQDIGMGMHNSMYHHHPHHHHHHHQHHQLQSAASSAGVVGVGLGGGGGGGPGPSLASGGSSTTSLSRKQSSKYEKRGVSITESQVSCQENTASEKSPVEHGGVALKA

SEQ ID NO: 37 - DNA, 1569 bp, Drosophila melanogaster
  CDS (1)..(1566)
atgaatcctttggtgctgggccacctactggcaccaaacaaactttttatacgattattgccccgtatttcaagccacaaagcattaagcaagccagtggaatttgccattcgcagctcgctagaaagtgtgataatcagcatgaatcagacggagcccgcccagctggcagatggggagcatctgagtggatacgccagcagcagcaacagcgtgcgctatctggacgaccggcatccgctggactaccttgacctgggcacggtgcacgccctcaacaccactgccatcaacacctcggatctgaatgagactgggagcaggccgctggacccggtgcttatcgataggttcctgagcaacagggcggtggacagcccctggtaccacatgctcatcagcatgtacggcgtgctaatcgtcttcggcgccctaggcaacaccctggttgttatagccgtcatccggaagcccatcatgcgcactgctcgcaatctgttcatcctcaacctggccatatcggtcaccatgccgctgaccttgatggagatcctgtccaagtactggccctacggctcctgctccatcctgtgcaaaacgattgccatgctgcaggcactttgtattttcgtgtcgacaatatccataacggccattgccttcgacagatatcaggtgatcgtgtaccccacgcgggacagcctgcagttcgtgggcgcggtgacgatcctggcggggatctgggcactggcactgctgctggcctcgccgctgttcgtctacaaggagctgatcaacacagacacgccggcactcctgcagcagatcggcctgcaggacacgatcccgtactgcattgaggactggccaagtcgcaacgggcgcttctactactcgatcttctcgctgtgcgtacaatacctggtgcccatcctgatcgtctcggtggcatacttcgggatctacaacaagctgaagagccgcatcaccgtggtggctgtgcaggcgtcctccgctcagcggaaggtggagcgggggcggcggatgaagcgcaccaactgcctactgatcagcatcgccatcatctttggcgtttcttggctgccgctgaactttttcaacctgtacgcggacatggagcgctcgccggtcactcagagcatgctagtccgctacgccatctgccacatgatcggcatgagctccgcctgctccaacccgttgctctacggctggctcaacgacaacttccgtaaagaatttcaagaactgctctgccgttgctcagacactaatgttgctcttaacggtcacacgacaggctgcaacgtccaggcggcggcgcgcaggcgtcgcaagttgggcgccgaactctccaaaggcgaactcaagctgctggggccaggcggcgcccagagcggtaccgccggcggggaaggcggtctggcggccaccgacttcatgaccggccaccacgagggcggactgcgcagcgccataaccgagtcggtggccctcacggaccacaaccccgtgccctcggaggtcaccaagctgatgccgcggtaa

SEQ ID NO: 38 - protein, 522 aa, Drosophila melanogaster
MNPLVLGHLLAPNKLFIRLLPRISSHKALSKPVEFAIRSSLESVIISMNQTEPAQLADGEHLSGYASSSNSVRYLDDRHPLDYLDLGTVHALNTTAINTSDLNETGSRPLDPVLIDRFLSNRAVDSPWYHMLISMYGVLIVFGALGNTLVVIAVIRKPIMRTARNLFILNLAISVTMPLTLMEILSKYWPYGSCSILCKTIAMLQALCIFVSTISITAIAFDRYQVIVYPTRDSLQFVGAVTILAGIWALALLLASPLFVYKELINTDTPALLQQIGLQDTIPYCIEDWPSRNGRFYYSIFSLCVQYLVPILIVSVAYFGIYNKLKSRITVVAVQASSAQRKVERGRRMKRTNCLLISIAIIFGVSWLPLNFFNLYADMERSPVTQSMLVRYAICHMIGMSSACSNPLLYGWLNDNFRKEFQELLCRCSDTNVALNGHTTGCNVQAAARRRRKLGAELSKGELKLLGPGGAQSGTAGGEGGLAATDFMTGHHEGGLRSAITESVALTDHNPVPSEVTKLMPR

SEQ ID NO: 39 - DNA, 540 bp, Drosophila melanogaster
  CDS (1)..(537)
atggagaacaccacaatgctggctaatattagcctaaatgcaaccagaaatgaggagaatatcacctcattcttcaccgacgaagagtggctggccatcaatggcactttgccgtggatagtgggattcttcttcggcgtcatcgccatcacgggattcttcggcaacctgctggtcatcctggtggtggtcttcaacaacaacatgcgctccaccaccaacctgatgattgtcaatctggctgccgctgatctgatgttcgtaatcctctgcattcccttcacggccaccgattacatggtgtactactggccatatggaaggttctggtgccgcagtgtccagtacctgattgtggtgaccgccttcgcctccatttacacgctggtgctgatgtccatcgatcggttcctggcggtggttcatcccattcgctcgcggatgatgaggacggagaacattaccctgattgccatcgtgactctgtggatcgtggtgctggtcgtttcggtgccagtggccttcacccacgacgtggtg

SEQ ID NO: 40 - protein, 179 aa, Drosophila melanogaster
MENTTMLANISLNATRNEENITSFFTDEEWLAINGTLPWIVGFFFGVIAITGFFGNLLVILVVVFNNNMRSTTNLMIVNLAAADLMFVILCIPFTATDYMVYYWPYGRFWCRSVQYLIVVTAFASIYTLVLMSIDRFLAVVHPIRSRMMRTENITLIAIVTLWIVVLVVSVPVAFTHDV

SEQ ID NO: 41 - DNA, 624 bp, Drosophila melanogaster
  CDS (1)..(621)
atgacgtatgcgcaatattcgattcccacaccgcacacgtttcttcatagttcccacaaccacattctcatcgatatccgcaggtggattacgatgcaaagaagaacatcacctacggcatgtgcaccttcacgacgaacgacttccttggtcaccttcttcatcagctcctacctgctgcccctgatgatcatcagcggtctctacatgcgcatgatcatgcggctctggcgccagggaaccggcgtccgcatgtccaaggagtcgcagcgcggtcgcaagcgggtcacccgactcgtcgtcgtggtggtcatcgccttcgcctcgctctggctgcctgtccagctcatcctgctgctcaagtcactggatgtcatcgagacgaacaccctcaccaagctagtcatccaggtcaccgcccagactctggcctacagcagctcgtgtatcaatccgctgctctacgccttcctctccgagaatttccggaaggccttctacaagacaatgcgaatgcacagcgcacctggaaagccaaggaccgcaactgccggcggttcaaaacccgtaattcatttccccggagccctgtgctgtgttgctgtctctgtgacgaggcactaa

SEQ ID NO: 42 - protein, 207 aa, Drosophila melanogaster
MTYAQYSIPTPHTFLHSSHNHILIDIRRWITMQRRTSPTACAPSRRTTSLVTFFISSYLLPLMIISGLYMRMIMRLWRQGTGVRMSKESQRGRKRVTRLVVVVVIAFASLWLPVQLILLLKSLDVIETNTLTKLVIQVTAQTLAYSSSCINPLLYAFLSENFRKAFYKTMRMHSAPGKPRTATAGGSKPVIHFPGALCCVAVSVTRH

SEQ ID NO: 43 - DNA, 1566 bp, Drosophila melanogaster
  CDS (1)..(1563)
atggccctgctccactacaccttcgaccagctcgagttgtacctagaatgggcgttcgcccagcacggagaggccacgccgattccctcgatccagccgtatccgggcgtgttcgtgggcgatctcagccagctgaaccgcttcaagcgacatgccttctccgccgtcgtgggcacgctcttcgtcctcgccttctgcggcaacctgagcaccctctatgtgaacagccgccggaagctgcggcccttcttccgggcctgcctcatctcactggcctgcagtgaccttgtctccagcatattctgcaccgtatcctacatggcccagttccaggcgcagtatctgcagctctggacaattggtggcttcatgtgcaagtttgtgccgttcataaccaccacgtcggtgttgtccggcagcctgaccctggtggccatcgccttggaccgctacttggccgtgatgcgacccgtgctggggttctggagtcccgacaaacgcttcagcactctgagcatgctgctcatctgggcatgttcgataggatcctctggcccgctgctgggcatctacgactaccgaaagatctatctcctggatgtggaggactccagcgaggagagcgaggaggtcgtgaccgctgtgcccgaggagctggtggtcaccgagctggaaatggttcacatgtgcctggccggggaccacgacgtcggactctactacgtcatactgttcaccctgatcttcctgccgtgcattgtgtccttcctatggctgaatgccgtgattgcgaggcagctctggctgcggagacactaccaccaggagcagcaggagcagcatcaggagcccaaggagggtcagtttaagaccatggcgaatggcggggatctcttgatgccctccaccttggtcagtgccatgggagtggccgttccctttgcgctggacaacactcccttgccgcccaagagtaccgtcaacgctcctggcaagaagaccactgctgctgctctagccagggaggccaggcaccgcaagatggtggtggtggtgctgctaatgatggcggttttcatatgccttcgactgcccgcctgggtcttcctgatcatgcggctttatggatcctactccgagcccatcgactggctcctgtacttcagcttcggcatactgaaccttttcagttgcgccctaaatcccatcttctacaccttcctcacccagaccattaggaccttgacgctggtgaaacacaaaattcagggatttcttggctgtccgcctggtaaagtgccggatggtatgcccacggatcagatggacaaaagcgggtgctgctgcggcctgaggccacccaccttcacatggaggtgccatcccagccgcgatagggcagcagccaccgtaatccgggatgtggaccagccagatccgccctccgaccaggtgcaaccggatccgagtagcctacggcggttcctcagctacaagcaggaggtgttcaccatttacaagcagtgcggtgactcgtcgagtgcgtcaatcgagtcctccgcctga

SEQ ID NO: 44 - protein, 521 aa, Drosophila melanogaster
MALLHYTFDQLELYLEWAFAQHGEATPIPSIQPYPGVFVGDLSQLNRFKRHAFSAVVGTLFVLAFCGNLSTLYVNSRRKLRPFFRACLISLACSDLVSSIFCTVSYMAQFQAQYLQLWTIGGFMCKFVPFITTTSVLSGSLTLVAIALDRYLAVMRPVLGFWSPDKRFSTLSMLLIWACSIGSSGPLLGIYDYRKIYLLDVEDSSEESEEVVTAVPEELVVTELEMVHMCLAGDHDVGLYYVILFTLIFLPCIVSFLWLNAVIARQLWLRRHYHQEQQEQHQEPKEGQFKTMANGGDLLMPSTLVSAMGVAVPFALDNTPLPPKSTVNAPGKKTTAAALAREARHRKMVVVVLLMMAVFICLRLPAWVFLIMRLYGSYSEPIDWLLYFSFGILNLFSCALNPIFYTFLTQTIRTLTLVKHKIQGFLGCPPGKVPDGMPTDQMDKSGCCCGLRPPTFTWRCHPSRDRAAATVIRDVDQPDPPSDQVQPDPSSLRRFLSYKQEVFTIYKQCGDSSSASIESSA

SEQ ID NO: 45 - DNA, 816 bp, Drosophila melanogaster
  CDS (1)..(813)
atggaggacgagtggggctcctttgatcgtctgcccagtgttccgagtgcctcgatggatttggagacggaaaacgaggtggtcagcaattggtccacactggccaacttcacgcgacttgtggctggtgccgctcctgaaatcgtcaactatacgctcaacatgatcgacgtgggtgtgggcatggccacggatatatccaatttgagcgttccggagcacgtgatggatcacgcacctcaactatcccgatccgggttgctgaaagtgtatgtcctggcggtaatggcactgttctctctgctgggcaacctgctgaccatctggaatatctacaaaacccgcatctcaagaagaaactcacggcacacgtggagtgctatctactcactgatgttccatctgtccatcgccgatgtcctggtcacctggttctgcattatcggggaggccgcgtggtgctacaccgtccagtggctggccaatgagctcacctgcaagctggtgaagctcttccagatgtttagcctctacctgagcacctatgtcctggtcctcatcggagtggaccgctggatagcggtcaagtatccgatgaagtcgctcaacatggccaagaggtgtcataggctacttggcggtacttacatcctgtcgctggtgctcagcttgccacagataggcacactgggtgatatattcctttgccctggcagtcagctggcggataagcctgaatgggactgcattaatgggcgtcactcgctttgttctacaactctagaacgactcggcttcagatttccggcaccctga

SEQ ID NO: 46 - protein, 271 aa, Drosophila melanogaster
MEDEWGSFDRLPSVPSASMDLETENEVVSNWSTLANFTRLVAGAAPEIVNYTLNMIDVGVGMATDISNLSVPEHVMDHAPQLSRSGLLKVYVLAVMALFSLLGNLLTIWNIYKTRISRRNSRHTWSAIYSLMFHLSIADVLVTWFCIIGEAAWCYTVQWLANELTCKLVKLFQMFSLYLSTYVLVLIGVDRWIAVKYPMKSLNMAKRCHRLLGGTYILSLVLSLPQIGTLGDIFLCPGSQLADKPEWDCINGRHSLCSTTLERLGFRFPAP

SEQ ID NO: 47 - DNA, 21 bp, Artificial Sequence
  Description of Artificial Sequence: Primer
aaggtcatcaaaatgctgatt

SEQ ID NO: 48 - DNA, 21 bp, Artificial Sequence
  Description of Artificial Sequence: Primer
attgtagcagctattgctcat

SEQ ID NO: 49 - DNA, 21 bp, Artificial Sequence
  Description of Artificial Sequence: Primer
cagctcgttcgattcggtcct

SEQ ID NO: 50 - DNA, 21 bp, Artificial Sequence
  Description of Artificial Sequence: Primer
gtgacagcggtcatagtccga

SEQ ID NO: 51 - DNA, 21 bp, Artificial Sequence
  Description of Artificial Sequence: Primer
atcgaggcatccacctatggc

SEQ ID NO: 52 - DNA, 21 bp, Artificial Sequence
  Description of Artificial Sequence: Primer
aggtgggcgcaggcatcgtag

SEQ ID NO: 53 - DNA, 23 bp, Artificial Sequence
  Description of Artificial Sequence: Primer
ttgctacgtagttctgaggaatc

SEQ ID NO: 54 - DNA, 21 bp, Artificial Sequence
  Description of Artificial Sequence: Primer
atgcaggtggagagcttcatg

SEQ ID NO: 55 - DNA, 21 bp, Artificial Sequence
  Description of Artificial Sequence: Primer
tggcagacgagtgcttcctga

SEQ ID NO: 56 - DNA, 20 bp, Artificial Sequence
  Description of Artificial Sequence: Primer
ggaccgctgaagttgaccag

SEQ ID NO: 57 - DNA, 21 bp, Artificial Sequence
  Description of Artificial Sequence: Primer
tggtctggtacctgctggtca

SEQ ID NO: 58 - DNA, 21 bp, Artificial Sequence
  Description of Artificial Sequence: Primer
gcgatgagccatttgaccagc

SEQ ID NO: 59 - DNA, 21 bp, Artificial Sequence
  Description of Artificial Sequence: Primer
gtgacccatgcgttcatcatc

SEQ ID NO: 60 - DNA, 19 bp, Artificial Sequence
  Description of Artificial Sequence: Primer
ctgcagcatgggcagaaag

SEQ ID NO: 61 - DNA, 21 bp, Artificial Sequence
  Description of Artificial Sequence: Primer
gcgatcacctggaagatctgc

SEQ ID NO: 62 - DNA, 21 bp, Artificial Sequence
  Description of Artificial Sequence: Primer
tggtgatgccaataggatacc

SEQ ID NO: 63 - DNA, 21 bp, Artificial Sequence
  Description of Artificial Sequence: Primer
ctgttgcacttcctggtctac

SEQ ID NO: 64 - DNA, 21 bp, Artificial Sequence
  Description of Artificial Sequence: Primer
acgcacagctccctgaatttc

SEQ ID NO: 65 - DNA, 20 bp, Artificial Sequence
  Description of Artificial Sequence: Primer
gaggagcacgatgtgagtgg

SEQ ID NO: 66 - DNA, 20 bp, Artificial Sequence
  Description of Artificial Sequence: Primer
cgtgtaaacggataattctg

SEQ ID NO: 67 - DNA, 20 bp, Artificial Sequence
  Description of Artificial Sequence: Primer
gtctggctgatacccagcta

SEQ ID NO: 68 - DNA, 21 bp, Artificial Sequence
  Description of Artificial Sequence: Primer
acgctcttgactttctcgaac

SEQ ID NO: 69 - DNA, 21 bp, Artificial Sequence
  Description of Artificial Sequence: Primer
ttcgcacaccagttctacgac

SEQ ID NO: 70 - DNA, 21 bp, Artificial Sequence
  Description of Artificial Sequence: Primer
gcgttcatgaagcagtaggtg

SEQ ID NO: 71 - DNA, 20 bp, Artificial Sequence
  Description of Artificial Sequence: Primer
tacatctgcatcggacgtgg

SEQ ID NO: 72 - DNA, 19 bp, Artificial Sequence
  Description of Artificial Sequence: Primer
cactatgccgcattgctcc

SEQ ID NO: 73 - DNA, 21 bp, Artificial Sequence
  Description of Artificial Sequence: Primer
gtgatctatgtggtgatgagg

SEQ ID NO: 74 - DNA, 21 bp, Artificial Sequence
  Description of Artificial Sequence: Primer
cctcgactgcacggtgctggc

SEQ ID NO: 75 - DNA, 21 bp, Artificial Sequence
  Description of Artificial Sequence: Primer
gtcatcgttctgggcaattca

SEQ ID NO: 76 - DNA, 21 bp, Artificial Sequence
  Description of Artificial Sequence: Primer
gatgctcatggccaccagcac

SEQ ID NO: 77 - DNA, 21 bp, Artificial Sequence
  Description of Artificial Sequence: Primer
aacgtactgcgagtgatcgtg

SEQ ID NO: 78 - DNA, 21 bp, Artificial Sequence
  Description of Artificial Sequence: Primer
aatggcaaaggtgacatcgtg

SEQ ID NO: 79 - DNA, 21 bp, Artificial Sequence
  Description of Artificial Sequence: Primer
atgtgccgcatcagcgagttc

SEQ ID NO: 80 - DNA, 21 bp, Artificial Sequence
  Description of Artificial Sequence: Primer
gttaccggtggctgtgaacac

SEQ ID NO: 81 - DNA, 21 bp, Artificial Sequence
  Description of Artificial Sequence: Primer
cagaaaccgctcaaggagacg

SEQ ID NO: 82 - DNA, 21 bp, Artificial Sequence
  Description of Artificial Sequence: Primer
cctcagacgagccgcagttag

SEQ ID NO: 83 - DNA, 19 bp, Artificial Sequence
  Description of Artificial Sequence: Primer
gcactggcactgctgctgg

SEQ ID NO: 84 - DNA, 20 bp, Artificial Sequence
  Description of Artificial Sequence: Primer
cacagccaccacggtgatgc

SEQ ID NO: 85 - DNA, 21 bp, Artificial Sequence
  Description of Artificial Sequence: Primer
ggcactttgccgtggatagtg

SEQ ID NO: 86 - DNA, 21 bp, Artificial Sequence
  Description of Artificial Sequence: Primer
gaaccgatcgatggacatcag

SEQ ID NO: 87 - DNA, 20 bp, Artificial Sequence
  Description of Artificial Sequence: Primer
catcagctcctacctgctgc

SEQ ID NO: 88 - DNA, 21 bp, Artificial Sequence
  Description of Artificial Sequence: Primer
cgtagagcagcggattgatac

SEQ ID NO: 89 - DNA, 21 bp, Artificial Sequence
  Description of Artificial Sequence: Primer
catctcactggcctgcagtga

SEQ ID NO: 90 - DNA, 20 bp, Artificial Sequence
  Description of Artificial Sequence: Primer
catgctcagagtcgacttcg

SEQ ID NO: 91 - DNA, 20 bp, Artificial Sequence
  Description of Artificial Sequence: Primer
gcggtaatggcactgttctc

SEQ ID NO: 92 - DNA, 21 bp, Artificial Sequence
  Description of Artificial Sequence: Primer
ggatactgtggagaaccggta